SEQUENCE LISTING (14 sequences)

SEQ ID NO: 1 - DNA, 3060 bp, Aspergillus fumigatus
atgagattcggttggctcgaggtggccgctctgacggccgcttctgtagccaatgcccaggtttgtgatgctttcccgtcattgtttcggatatagttgacaatagtcatggaaataatcaggaattggctttctctccaccattctacccttcgccttgggctgatggccagggagagtgggcagatgcccatcgacgcgccgtcgagatcgtttctcagatgacactggcggagaaggttaaccttacaacgggtactgggtgggttgcgacttttttgttgacagtgagctttcttcactgaccatctacacagatgggaaatggaccgatgcgtcggtcaaaccggcagcgttcccaggtaagcttgcaattctgcaacaacgtgcaagtgtagttgctaaaacgcggtggtgcagacttggtatcaactggggtctttgtggccaggattcccctttgggtatccgtttctgtgagctatacccgcggagtctttcagtccttgtattatgtgctgatgattgtctctgtatagctgacctcaactccgccttccctgctggtactaatgtcgccgcgacatgggacaagacactcgcctaccttcgtggcaaggccatgggtgaggaattcaacgacaagggcgtggacattttgctggggcctgctgctggtcctctcggcaaatacccggacggcggcagaatctgggaaggcttctctcctgatccggttctcactggtgtacttttcgccgaaactatcaagggtatccaagacgcgggtgtgattgctactgccaagcattacattctgaatgaacaggagcatttccgacaggttggcgaggcccagggatatggttacaacatcacggagacgatcagctccaacgtggatgacaagaccatgcacgagttgtacctttggtgagtagttgacactgcaaatgaggaccttgattgatttgactgacctggaatgcaggccctttgcagatgctgtgcgcggtaagattttccgtagacttgacctcgcgacgaagaaatcgctgacgaaccatcgtagctggcgttggcgctgtcatgtgttcctacaatcaaatcaacaacagctacggttgtcaaaacagtcaaactctcaacaagctcctcaaggctgagctgggcttccaaggcttcgtcatgagtgactggagcgctcaccacagcggtgtcggcgctgccctcgctgggttggatatgtcgatgcctggagacatttccttcgacgacggactctccttctggggcacgaacctaactgtcagtgttcttaacggcaccgttccagcctggcgtgtcgatgacatggctgttcgtatcatgaccgcgtactacaaggttggtcgtgaccgtcttcgtattccccctaacttcagctcctggacccgggatgagtacggctgggagcattctgctgtctccgagggagcctggaccaaggtgaacgacttcgtcaatgtgcagcgcagtcactctcagatcatccgtgagattggtgccgctagtacagtgctcttgaagaacacgggtgctcttcctttgaccggcaaggaggttaaagtgggtgttctcggtgaagacgctggttccaacccgtggggtgctaacggctgccccgaccgcggctgtgataacggcactcttgctatggcctggggtagtggtactgccaacttcccttaccttgtcacccccgagcaggctatccagcgagaggtcatcagcaacggcggcaatgtctttgctgtgactgataacggggctctcagccagatggcagatgttgcatctcaatccaggtgagtgcgggctcttagaaaaagaacgttctctgaatgaagttttttaaccattgcgaacagcgtgtctttggtgtttgtcaacgccgactctggagagggtttcatcagtgtcgacggcaacgagggtgaccgcaaaaatctcactctgtggaagaacggcgaggccgtcattgacactgttgtcagccactgcaacaacacgattgtggttattcacagtgttgggcccgtcttgatcgaccggtggtatgataaccccaacgtcactgccatcatctgggccggcttgcccggtcaggagagtggcaactccctggtcgacgtgctctatggccgcgtcaaccccagcgccaagaccccgttcacctggggcaagactcgggagtcttacggggctcccttgctcaccgagcctaacaatggcaatggtgctccccaggatgatttcaacgagggcgtcttcattgactaccgtcactttgacaagcgcaatgagacccccatttatgagtttggccatggcttgagctacaccacctttggttactctcaccttcgggttcaggccctcaatagttcgagttcggcatatgtcccgactagcggagagaccaagcctgcgccaacctatggtgagatcggtagtgccgccgactacctgtatcccgagggtctcaaaagaattaccaagtttatttacccttggctcaactcgaccgacctcgaggattcttctgacgacccgaactacggctgggaggactcggagtacattcccgaaggcgctagggatgggtctcctcaacccctcctgaaggctggcggcgctcctggtggtaaccctaccctttatcaggatcttgttagggtgtcggccaccataaccaacactggtaacgtcgccggttatgaagtccctcaattggtgagtgacccgcatgttccttgcgttgcaatttggctaactcgcttctagtatgtttcactgggcggaccgaacgagcctcgggtcgttctgcgcaagttcgaccgaatcttcctggctcctggggagcaaaaggtttggaccacgactcttaaccgtcgtgatctcgccaattgggatgtggaggctcaggactgggtcatcacaaagtaccccaagaaagtgcacgtcggcagctcctcgcgtaagctgcctctgagagcgcctctgccccgtgtctactag

SEQ ID NO: 2 - protein, 863 aa, Aspergillus fumigatus
MRFGWLEVAALTAASVANAQELAFSPPFYPSPWADGQGEWADAHRRAVEIVSQMTLAEKVNLTTGTGWEMDRCVGQTGSVPRLGINWGLCGQDSPLGIRFSDLNSAFPAGTNVAATWDKTLAYLRGKAMGEEFNDKGVDILLGPAAGPLGKYPDGGRIWEGFSPDPVLTGVLFAETIKGIQDAGVIATAKHYILNEQEHFRQVGEAQGYGYNITETISSNVDDKTMHELYLWPFADAVRAGVGAVMCSYNQINNSYGCQNSQTLNKLLKAELGFQGFVMSDWSAHHSGVGAALAGLDMSMPGDISFDDGLSFWGTNLTVSVLNGTVPAWRVDDMAVRIMTAYYKVGRDRLRIPPNFSSWTRDEYGWEHSAVSEGAWTKVNDFVNVQRSHSQIIREIGAASTVLLKNTGALPLTGKEVKVGVLGEDAGSNPWGANGCPDRGCDNGTLAMAWGSGTANFPYLVTPEQAIQREVISNGGNVFAVTDNGALSQMADVASQSSVSLVFVNADSGEGFISVDGNEGDRKNLTLWKNGEAVIDTVVSHCNNTIVVIHSVGPVLIDRWYDNPNVTAIIWAGLPGQESGNSLVDVLYGRVNPSAKTPFTWGKTRESYGAPLLTEPNNGNGAPQDDFNEGVFIDYRHFDKRNETPIYEFGHGLSYTTFGYSHLRVQALNSSSSAYVPTSGETKPAPTYGEIGSAADYLYPEGLKRITKFIYPWLNSTDLEDSSDDPNYGWEDSEYIPEGARDGSPQPLLKAGGAPGGNPTLYQDLVRVSATITNTGNVAGYEVPQLYVSLGGPNEPRVVLRKFDRIFLAPGEQKVWTTTLNRRDLANWDVEAQDWVITKYPKKVHVGSSSRKLPLRAPLPRVY

SEQ ID NO: 3 - DNA, 3060 bp, Aspergillus fumigatus
atgagattcggttggctcgaggtggccgctctgacggccgcttctgtagccaatgcccaggtttgtgatgctttcccgtcattgtttcggatatagttgacaatagtcatggaaataatcaggaattggctttctctccaccattctacccttcgccttgggctgatggccagggagagtgggcagatgcccatcgacgcgccgtcgagatcgtttctcagatgacactggcggagaaggttaaccttacaacgggtactgggtgggttgcgacttttttgttgacagtgagctttcttcactgaccatctacacagatgggaaatggaccgatgcgtcggtcaaaccggcagcgttcccaggtaagcttgcaattctgcaacaacgtgcaagtgtagttgctaaaacgcggtggtgcagacttggtatcaactggggtctttgtggccaggattcccctttgggtatccgtgactgtgagctatacccgcggagtctttcagtccttgtattatgtgctgatgattgtctctgtatagctgacctcaactccgccttccctgctggtactaatgtcgccgcgacatgggacaagacactcgcctaccttcgtggcaaggccatgggtgaggaattcaacgacaagggcgtggacattttgctggggcctgctgctggtcctctcggcaaatacccggacggcggcagaatctgggaaggcttctctcctgatccggttctcactggtgtacttttcgccgaaactatcaagggtatccaagacgcgggtgtgattgctactgccaagcattacattctgaatgaacaggagcatttccgacaggttggcgaggcccagggatatggttacaacatcacggagacgatcagctccaacgtggatgacaagaccatgcacgagttgtacctttggtgagtagttgacactgcaaatgaggaccttgattgatttgactgacctggaatgcaggccctttgcagatgctgtgcgcggtaagattttccgtagacttgacctcgcgacgaagaaatcgctgacgaaccatcgtagctggcgttggcgctgtcatgtgttcctacaatcaaatcaacaacagctacggttgtcaaaacagtcaaactctcaacaagctcctcaaggctgagctgggcttccaaggcttcgtcatgagtgactggggcgctcaccacagcggtgtcggcgctgccctcgctgggttggatatgtcgatgcctggagacatttccttcgacgacggactctccttctggggcacgaacctaactgtcagtgttcttaacggcaccgttccagcctggcgtgtcgatgacatggctgttcgtatcatgaccgcgtactacaaggttggtcgtgaccgtcttcgtattccccctaacttcagctcctggacccgggatgagtacggctgggagcattctgctgtctccgagggagcctggaccaaggtgaacgacttcgtcaatgtgcagcgcagtcactctcagatcatccgtgagattggtgccgctagtacagtgctcttgaagaacacgggtgctcttcctttgaccggcaaggaggttaaagtgggtgttctcggtgaagacgctggttccaacccgtggggtgctaacggctgccccgaccgcggctgtgataacggcactcttgctatggcctggggtagtggtactgccgagttcccttaccttgtcacccccgagcaggctatccagcgagaggtcatcagcaacggcggcaatgtctttgctgtgactgataacggggctctcagccagatggcagatgttgcatctcaatccaggtgagtgcgggctcttagaaaaagaacgttctctgaatgaagttttttaaccattgcgaacagcgtgtctttggtgtttgtcaacgccgactctggagagggttacatcagtgtcgacggcaacgagggtgaccgcaaaaatctcactctgtggaagaacggcgaggccgtcattgacactgttgtcagccactgcaacaacacgattgtggttattcacagtgttgggcccgtcttgatcgaccggtggtatgataaccccaacgtcactgccatcatctgggccggcttgcccggtcaggagagtggcaactccctggtcgacgtgctctatggccgcgtcaaccccagcgccaagaccccgttcacctggggcaagactcgggagtcttacggggctcccttgctcaccgagcctaacaatggcaatggtgctccccaggatgatttcaacgagggcgtcttcattgactaccgtcactttgacaagcgcaatgagacccccatttatgagtttggccatggcttgagctacaccacctttggttactctcaccttcgggttcaggccctcaatagttcgagttcggcatatgtcccgactagcggagagaccaagcctgcgccaacctatggtgagatcggtagtgccgccgactacctgtatcccgagggtctcaaaagaattaccaagtttatttacccttggctcaactcgaccgacctcgaggattcttctgacgacccgaactacggctgggaggactcggagtacattcccgaaggcgctagggatgggtctcctcaacccctcctgaaggctggcggcgctcctggtggtaaccctaccctttatcaggatcttgttagggtgtcggccaccataaccaacactggtaacgtcgccggttatgaagtccctcaattggtgagtgacccgcatgttccttgcgttgcaatttggctaactcgcttctagtatgtttcactgggcggaccgaacgagcctcgggtcgttctgcgcaagttcgaccgaatcttcctggctcctggggagcaaaaggtttggaccacgactcttaaccgtcgtgatctcgccaattgggatgtggaggctcaggactgggtcatcacaaagtaccccaagaaagtgcacgtcggcagctcctcgcgtaagctgcctctgagagcgcctctgccccgtgtctactag

SEQ ID NO: 4 - protein, 863 aa, Aspergillus fumigatus
MRFGWLEVAALTAASVANAQELAFSPPFYPSPWADGQGEWADAHRRAVEIVSQMTLAEKVNLTTGTGWEMDRCVGQTGSVPRLGINWGLCGQDSPLGIRDSDLNSAFPAGTNVAATWDKTLAYLRGKAMGEEFNDKGVDILLGPAAGPLGKYPDGGRIWEGFSPDPVLTGVLFAETIKGIQDAGVIATAKHYILNEQEHFRQVGEAQGYGYNITETISSNVDDKTMHELYLWPFADAVRAGVGAVMCSYNQINNSYGCQNSQTLNKLLKAELGFQGFVMSDWGAHHSGVGAALAGLDMSMPGDISFDDGLSFWGTNLTVSVLNGTVPAWRVDDMAVRIMTAYYKVGRDRLRIPPNFSSWTRDEYGWEHSAVSEGAWTKVNDFVNVQRSHSQIIREIGAASTVLLKNTGALPLTGKEVKVGVLGEDAGSNPWGANGCPDRGCDNGTLAMAWGSGTAEFPYLVTPEQAIQREVISNGGNVFAVTDNGALSQMADVASQSSVSLVFVNADSGEGYISVDGNEGDRKNLTLWKNGEAVIDTVVSHCNNTIVVIHSVGPVLIDRWYDNPNVTAIIWAGLPGQESGNSLVDVLYGRVNPSAKTPFTWGKTRESYGAPLLTEPNNGNGAPQDDFNEGVFIDYRHFDKRNETPIYEFGHGLSYTTFGYSHLRVQALNSSSSAYVPTSGETKPAPTYGEIGSAADYLYPEGLKRITKFIYPWLNSTDLEDSSDDPNYGWEDSEYIPEGARDGSPQPLLKAGGAPGGNPTLYQDLVRVSATITNTGNVAGYEVPQLYVSLGGPNEPRVVLRKFDRIFLAPGEQKVWTTTLNRRDLANWDVEAQDWVITKYPKKVHVGSSSRKLPLRAPLPRVY

SEQ ID NO: 5 - DNA, 1599 bp, Aspergillus fumigatus
atgctggcctccaccttctcctaccgcatgtacaagaccgcgctcatcctggccgcccttctgggctctggccaggctcagcaggtcggtacttcccaggcggaagtgcatccgtccatgacctggcagagctgcacggctggcggcagctgcaccaccaacaacggcaaggtggtcatcgacgcgaactggcgttgggtgcacaaagtcggcgactacaccaactgctacaccggcaacacctgggacacgactatctgccctgacgatgcgacctgcgcatccaactgcgcccttgagggtgccaactacgaatccacctatggtgtgaccgccagcggcaattccctccgcctcaacttcgtcaccaccagccagcagaagaacattggctcgcgtctgtacatgatgaaggacgactcgacctacgagatgtttaagctgctgaaccaggagttcaccttcgatgtcgatgtctccaacctcccctgcggtctcaacggtgctctgtactttgtcgccatggacgccgacggtggcatgtccaagtacccaaccaacaaggccggtgccaagtacggtactggatactgtgactcgcagtgccctcgcgacctcaagttcatcaacggtcaggccaacgtcgaagggtggcagccctcctccaacgatgccaatgcgggtaccggcaaccacgggtcctgctgcgcggagatggatatctgggaggccaacagcatctccacggccttcaccccccatccgtgcgacacgcccggccaggtgatgtgcaccggtgatgcctgcggtggcacctacagctccgaccgctacggcggcacctgcgaccccgacggatgtgatttcaactccttccgccagggcaacaagaccttctacggccctggcatgaccgtcgacaccaagagcaagtttaccgtcgtcacccagttcatcaccgacgacggcacctccagcggcaccctcaaggagatcaagcgcttctacgtgcagaacggcaaggtgatccccaactcggagtcgacctggaccggcgtcagcggcaactccatcaccaccgagtactgcaccgcccagaagagcctgttccaggaccagaacgtcttcgaaaagcacggcggcctcgagggcatgggtgctgccctcgcccagggtatggttctcgtcatgtccctgtgggatgatcactcggccaacatgctctggctcgacagcaactacccgaccactgcctcttccaccactcccggcgtcgcccgtggtacctgcgacatctcctccggcgtccctgcggatgtcgaggcgaaccaccccgacgcctacgtcgtctactccaacatcaaggtcggccccatcggctcgaccttcaacagcggtggctcgaaccccggtggcggaaccaccacgacaactaccacccagcctactaccaccacgaccacggctggaaaccctggcggcaccggagtcgcacagcactatggccagtgtggtggaatcggatggaccggacccacaacctgtgccagcccttatacctgccagaagctgaatgattattactctcagtgcctgtag

SEQ ID NO: 6 - protein, 532 aa, Aspergillus fumigatus
MLASTFSYRMYKTALILAALLGSGQAQQVGTSQAEVHPSMTWQSCTAGGSCTTNNGKVVIDANWRWVHKVGDYTNCYTGNTWDTTICPDDATCASNCALEGANYESTYGVTASGNSLRLNFVTTSQQKNIGSRLYMMKDDSTYEMFKLLNQEFTFDVDVSNLPCGLNGALYFVAMDADGGMSKYPTNKAGAKYGTGYCDSQCPRDLKFINGQANVEGWQPSSNDANAGTGNHGSCCAEMDIWEANSISTAFTPHPCDTPGQVMCTGDACGGTYSSDRYGGTCDPDGCDFNSFRQGNKTFYGPGMTVDTKSKFTVVTQFITDDGTSSGTLKEIKRFYVQNGKVIPNSESTWTGVSGNSITTEYCTAQKSLFQDQNVFEKHGGLEGMGAALAQGMVLVMSLWDDHSANMLWLDSNYPTTASSTTPGVARGTCDISSGVPADVEANHPDAYVVYSNIKVGPIGSTFNSGGSNPGGGTTTTTTTQPTTTTTTAGNPGGTGVAQHYGQCGGIGWTGPTTCASPYTCQKLNDYYSQCL

SEQ ID NO: 7 - DNA, 1713 bp, Aspergillus fumigatus
atgaagcaccttgcatcttccatcgcattgactctactgttgcctgccgtgcaggcccagcagaccgtatggggccaatgtatgttctggctgtcactggaataagactgtatcaactgctgatatgcttctaggtggcggccaaggctggtctggcccgacgagctgtgttgccggcgcagcctgtagcacactgaatccctgtatgttagatatcgtcctgagtggagacttatactgacttccttagactacgctcagtgtatcccgggagccaccgcgacgtccaccaccctcacgacgacgacggcggcgacgacgacatcccagaccaccaccaaacctaccacgactggtccaactacatccgcacccaccgtgaccgcatccggtaaccctttcagcggctaccagctgtatgccaacccctactactcctccgaggtccatactctggccatgccttctctgcccagctcgctgcagcccaaggctagtgctgttgctgaagtgccctcatttgtttggctgtaagtggccttatcccaatactgagaccaactctctgacagtcgtagcgacgttgccgccaaggtgcccactatgggaacctacctggccgacattcaggccaagaacaaggccggcgccaaccctcctatcgctggtatcttcgtggtctacgacttgccggaccgtgactgcgccgctctggccagtaatggcgagtactcaattgccaacaacggtgtggccaactacaaggcgtacattgacgccatccgtgctcagctggtgaagtactctgacgttcacaccatcctcgtcatcggtaggccgtacacctccgttgcgcgccgcctttctctgacatcttgcagaacccgacagcttggccaacctggtgaccaacctcaacgtcgccaaatgcgccaatgcgcagagcgcctacctggagtgtgtcgactatgctctgaagcagctcaacctgcccaacgtcgccatgtacctcgacgcaggtatgcctcacttcccgcattctgtatcccttccagacactaactcatcaggccatgcgggctggctcggatggcccgccaacttgggccccgccgcaacactcttcgccaaagtctacaccgacgcgggttcccccgcggctgttcgtggcctggccaccaacgtcgccaactacaacgcctggtcgctcagtacctgcccctcctacacccagggagaccccaactgcgacgagaagaagtacatcaacgccatggcgcctcttctcaaggaagccggcttcgatgcccacttcatcatggatacctgtaagtgcttattccaatcgccgatgtgtgccgactaatcaatgtttcagcccggaatggcgtccagcccacgaagcaaaacgcctggggtgactggtgcaacgtcatcggcaccggcttcggtgttcgcccctcgactaacaccggcgatccgctccaggatgcctttgtgtggatcaagcccggtggagagagtgatggcacgtccaactcgacttccccccggtatgacgcgcactgcggatatagtgatgctctgcagcctgctcctgaggctggtacttggttccaggtatgtcatccattagccagatgagggataagtgactgacggacctaggcctactttgagcagcttctgaccaacgctaacccgtccttttaa

SEQ ID NO: 8 - protein, 454 aa, Aspergillus fumigatus
MKHLASSIALTLLLPAVQAQQTVWGQCGGQGWSGPTSCVAGAACSTLNPYYAQCIPGATATSTTLTTTTAATTTSQTTTKPTTTGPTTSAPTVTASGNPFSGYQLYANPYYSSEVHTLAMPSLPSSLQPKASAVAEVPSFVWLDVAAKVPTMGTYLADIQAKNKAGANPPIAGIFVVYDLPDRDCAALASNGEYSIANNGVANYKAYIDAIRAQLVKYSDVHTILVIEPDSLANLVTNLNVAKCANAQSAYLECVDYALKQLNLPNVAMYLDAGHAGWLGWPANLGPAATLFAKVYTDAGSPAAVRGLATNVANYNAWSLSTCPSYTQGDPNCDEKKYINAMAPLLKEAGFDAHFIMDTSRNGVQPTKQNAWGDWCNVIGTGFGVRPSTNTGDPLQDAFVWIKPGGESDGTSNSTSPRYDAHCGYSDALQPAPEAGTWFQAYFEQLLTNANPSF

SEQ ID NO: 9 - DNA, 835 bp, Penicillium sp. emersonii
atgctgtcttcgacgactcgcaccctcgcctttacaggccttgcgggccttctgtccgctcccctggtcaaggcccatggctttgtccagggcattgtcatcggtgaccaattgtaagtccctctcttgcagttctgtcgattaactgctggactgcttgcttgactccctgctgactcccaacagctacagcgggtacatcgtcaactcgttcccctacgaatccaacccaccccccgtcatcggctgggccacgaccgccaccgacctgggcttcgtcgacggcacaggataccaaggcccggacatcatctgccaccggaatgcgacgcccgcgccgctgacagcccccgtggccgccggcggcaccgtcgagctgcagtggacgccgtggccggacagccaccacggacccgtcatcacctacctggcgccgtgcaacggcaactgctcgaccgtcgacaagacgacgctggagttcttcaagatcgaccagcagggcctgatcgacgacacgagcccgccgggcacctgggcgtcggacaacctcatcgccaacaacaatagctggaccgtcaccattcccaacagcgtcgcccccggcaactacgtcctgcgccacgagatcatcgccctgcactcggccaacaacaaggacggcgcccagaactacccccagtgcatcaacatcgaggtcacgggcggcggctccgacgcgcctgagggtactctgggcgaggatctctaccatgacaccgacccgggcattctggtcgacatttacgagcccattgcgacgtataccattccggggccgcctgagccgacgttctag

SEQ ID NO: 10 - protein, 253 aa, Penicillium sp. emersonii
MLSSTTRTLAFTGLAGLLSAPLVKAHGFVQGIVIGDQFYSGYIVNSFPYESNPPPVIGWATTATDLGFVDGTGYQGPDIICHRNATPAPLTAPVAAGGTVELQWTPWPDSHHGPVITYLAPCNGNCSTVDKTTLEFFKIDQQGLIDDTSPPGTWASDNLIANNNSWTVTIPNSVAPGNYVLRHEIIALHSANNKDGAQNYPQCINIEVTGGGSDAPEGTLGEDLYHDTDPGILVDIYEPIATYTIPGPPEPTF

SEQ ID NO: 11 - DNA, 1415 bp, Aspergillus fumigatus
atggtccatctatcttcattggcagcagccctggctgctctgcctctgtatgtttacccactcacgagaggaggaacagctttgacattgctatagtgtatatggagctggcctgaacacagcagccaaagccaaaggactaaagtactttggttccgccacggacaatccagagctcacggactctgcgtatgtcgcgcaactgagcaacaccgatgattttggtcaaatcacacccggaaactccatgaaggtttgcttacgtctgcctccctggagcattgcctcaaaagctaattggttgttttgtttggatagtgggatgccaccgagccttctcagaattctttttcgttcgcaaatggagacgccgtggtcaatctggcgaacaagaatggccagctgatgcgatgccatactctggtctggcacagtcagctaccgaactggggtatgtaaacgtcttgtctattctcaaatactctctaacagttgacagtctctagcgggtcatggaccaatgcgacccttttggcggccatgaagaatcatatcaccaatgtggttactcactacaaggggaagtgctacgcctgggatgttgtcaatgaaggtttgttgctccatctatcctcaatagttcttttgaaactgacaagcctgtcaatctagccctgaacgaggacggtactttccgtaactctgtcttctaccagatcatcggcccagcatacattcctattgcgttcgccacggctgctgccgcagatcccgacgtgaaactctactacaacgactacaacattgaatactcaggcgccaaagcgactgctgcgcagaatatcgtcaagatgatcaaggcctacggcgcgaagatcgacggcgtcggcctccaggcacactttatcgtcggcagcactccgagtcaatcggatctgacgaccgtcttgaagggctacactgctctcggcgttgaggtggcctataccgaacttgacatccgcatgcagctgccctcgaccgccgcaaagctggcccagcagtccactgacttccaaggcgtggccgcagcatgcgttagcaccactggctgcgtgggtgtcactatctgggactggaccgacaagtactcctgggtccccagcgtgttccaaggctacggcgccccattgccttgggatgagaactatgtgaagaagccagcgtacgatggcctgatggcgggtcttggagcaagcggctccggcaccacaacgaccactactactacttctactacgacaggaggtacggaccctactggagtcgctcagaaatggggacagtgtggcggtattggctggaccgggccaacaacttgtgtcagtggtaccacttgccaaaagctgaatgactggtactcacagtgcctgtaa

SEQ ID NO: 12 - protein, 397 aa, Aspergillus fumigatus
MVHLSSLAAALAALPLVYGAGLNTAAKAKGLKYFGSATDNPELTDSAYVAQLSNTDDFGQITPGNSMKWDATEPSQNSFSFANGDAVVNLANKNGQLMRCHTLVWHSQLPNWVSSGSWTNATLLAAMKNHITNVVTHYKGKCYAWDVVNEALNEDGTFRNSVFYQIIGPAYIPIAFATAAAADPDVKLYYNDYNIEYSGAKATAAQNIVKMIKAYGAKIDGVGLQAHFIVGSTPSQSDLTTVLKGYTALGVEVAYTELDIRMQLPSTAAKLAQQSTDFQGVAAACVSTTGCVGVTIWDWTDKYSWVPSVFQGYGAPLPWDENYVKKPAYDGLMAGLGASGSGTTTTTTTTSTTTGGTDPTGVAQKWGQCGGIGWTGPTTCVSGTTCQKLNDWYSQCL

SEQ ID NO: 13 - DNA, 2376 bp, Aspergillus fumigatus
atggcggttgccaaatctattgctgccgtgctggtagcactgttgcctggtgcgcttgctcaggcgaatacaagctatgttgattacaatgtggaggcgaatccggatctcacccctcagtcggtcgctacgattgacctgtcctttcccgactgcgagaatggaccgctcagcaagactctcgtttgcgacacgtcggctcggccgcatgaccgagctgctgccctggtttccatgttcaccttcgaggagctggtgaacaacacaggcaacactagccctggtgttccaagacttggtctccctccgtaccaagtatggagcgaggctctccatggacttgaccgcgccaacttcacaaacgagggagagtacagctgggccacctcgttccccatgcctatcctgacaatgtcggccttgaaccgaaccctgatcaaccagatcgcgaccatcatcgcaactcaaggacgagctttcaataacgttgggcggtatgggctggacgtgtacgccccgaatataaatgcattcagatcggctatgtggggaagaggtcaagagacccccggagaagacgcttactgcctggcatcggcgtatgcgtacgagtatatcactggcatccagggtggtgttgatccggaacacctcaagttggtggccactgccaaacactatgcgggctacgatcttgagaactgggacggtcactcccgtttgggcaacgatatgaacattacacagcaggaactttccgaatactacacccctcagttccttgttgcagccagagacgccaaagtgcacagtgtcatgtgctcctacaacgcggtaaatggggtgcccagctgcgcaaactcgttcttcctccagaccctcctccgtgacacattcggcttcgtcgaggatggttatgtatccagcgactgcgactcggcgtacaatgtctggaacccgcacgagtttgcggccaacatcacgggggccgctgcagactctatccgggcggggacggacattgattgcggcactacttatcaatactatttcggcgaagcctttgacgagcaagaggtcacccgtgcagaaatcgaaagaggtgtgatccgcctgtacagcaacttggtgcgtctcggctatttcgatggcaatggaagcgtgtatcgggacctgacgtggaatgatgtcgtgaccacggatgcctggaatatctcatacgaagccgctgtagaaggcattgtcctactgaagaacgatggaaccttgcctctcgccaagtcggtccgcagtgttgcattgattgggccctggatgaatgtgacgactcagcttcagggcaactactttggaccggcgccttatctgattagtccgttgaatgccttccagaattctgacttcgacgtgaactacgctttcggcacgaacatttcatcccactccacagatgggttttccgaggcgttgtctgctgcgaagaaatccgacgtcatcatattcgcgggcgggattgacaacactttggaagcagaagccatggatcgcatgaatatcacatggcccggcaatcagctacagctcatcgaccagttgagccaactcggcaaaccgctgatcgtcctccagatgggcggcggccaagtcgactcctcctcgctcaagtccaacaagaatgtcaactccctgatctggggtggataccccggacaatccggcgggcaggctctcctagacatcatcaccggcaagcgcgcccccgccggccgactcgtggtcacgcagtacccggccgaatacgcaacccagttccccgccaccgacatgagcctgcggcctcacggcaataatcccggccagacctacatgtggtacaccggcacccccgtctacgagtttggccacgggctcttctacacgaccttccacgcctccctccctggcaccggcaaggacaagacctccttcaacatccaagacctcctcacgcagccgcatccgggcttcgcaaacgtcgagcaaatgcctttgctcaacttcaccgtgacgatcaccaataccggcaaggtcgcttccgactacactgctatgctcttcgcgaacaccaccgcgggacctgctccatacccgaacaagtggctcgtcggcttcgaccggctggcgagcctggaaccgcacaggtcgcagactatgaccatccccgtgactatcgacagcgtggctcgtacggatgaggccggcaatcgggttctctacccgggaaagtacgagttggccctgaacaatgagcggtcggttgtccttcagtttgtgctgacaggccgagaggctgtgattttcaagtggcctgtagagcagcagcagatttcgtctgcg

SEQ ID NO: 14 - protein, 792 aa, Aspergillus fumigatus
MAVAKSIAAVLVALLPGALAQANTSYVDYNVEANPDLTPQSVATIDLSFPDCENGPLSKTLVCDTSARPHDRAAALVSMFTFEELVNNTGNTSPGVPRLGLPPYQVWSEALHGLDRANFTNEGEYSWATSFPMPILTMSALNRTLINQIATIIATQGRAFNNVGRYGLDVYAPNINAFRSAMWGRGQETPGEDAYCLASAYAYEYITGIQGGVDPEHLKLVATAKHYAGYDLENWDGHSRLGNDMNITQQELSEYYTPQFLVAARDAKVHSVMCSYNAVNGVPSCANSFFLQTLLRDTFGFVEDGYVSSDCDSAYNVWNPHEFAANITGAAADSIRAGTDIDCGTTYQYYFGEAFDEQEVTRAEIERGVIRLYSNLVRLGYFDGNGSVYRDLTWNDVVTTDAWNISYEAAVEGIVLLKNDGTLPLAKSVRSVALIGPWMNVTTQLQGNYFGPAPYLISPLNAFQNSDFDVNYAFGTNISSHSTDGFSEALSAAKKSDVIIFAGGIDNTLEAEAMDRMNITWPGNQLQLIDQLSQLGKPLIVLQMGGGQVDSSSLKSNKNVNSLIWGGYPGQSGGQALLDIITGKRAPAGRLVVTQYPAEYATQFPATDMSLRPHGNNPGQTYMWYTGTPVYEFGHGLFYTTFHASLPGTGKDKTSFNIQDLLTQPHPGFANVEQMPLLNFTVTITNTGKVASDYTAMLFANTTAGPAPYPNKWLVGFDRLASLEPHRSQTMTIPVTIDSVARTDEAGNRVLYPGKYELALNNERSVVLQFVLTGREAVIFKWPVEQQQISSA

(19) United States
(12) Patent Application Publication
(10) Pub. No.: US 2018/0051306 A1
(43) Pub. Date: Feb. 22, 2018

(54) Enzymatic Hydrolysis with Hemicellulolytic Enzymes

(71) Applicants: Novozymes A/S, Bagsvaerd (DK); Beta Renewables Spa, Tortona (IT)

(72) Inventors: Jesper Frickmann, Raleigh, NC (US); Johan Belfrage, Cupar (GB); Eric Abbate, Vacaville, CA (US); David Osborn, Sacramento, CA (US); Geoffrey Moxley, Raleigh, NC (US); Daniele Riva, Prela Imperia (IT); Piero Otonello, Milano (IT); Simone Ferrero, Tortona (IT); Micol Purrotti, Condove (IT); Stefano Paravisi, Tortona (IT); Chiara Prefumo, Genova (IT)

(73) Assignees: Novozymes A/S, Bagsvaerd (DK); Beta Renewables Spa, Tortona (IT)

(21) Appl. No.: 15/557,305

(22) PCT Filed: Mar. 11, 2016
(86) PCT No.: PCT/US2016/022091

§ 371 Date: Sep. 11, 2017

Related U.S. Application Data
(60) Provisional application No. 62/132,116, filed on Mar. 12, 2015.

(51) Int. Cl. C12P 19/14; C12P 19/02

(52) U.S. Cl. CPC C12P 19/14; C12P 19/02; C12Y 302/01; C12P 2203/00; C12Y 302/01091; C12Y 302/01008; C12Y 302/01037; C12Y 302/01021

(57) ABSTRACT

The invention relates to processes of closing an observed gap in sugar yield between pure batch hydrolysis and a multi-stage hydrolysis containing a continuous reactor. Enzyme compositions containing varied ratios of cellulolytic composition and a hemicellulolytic composition preconditioning are used in multi-stage hydrolysis processes containing a continuous reactor in order to close the gap. Such enzyme compositions are useful in multi-stage saccharification of a lignocellulosic material, fermentation processes following multi-stage saccharification of a lignocellulosic material and in improving a glucose or xylose yield in multi-stage saccharification of a lignocellulosic material.

### REFERENCE TO A SEQUENCE LISTING

This application contains a Sequence Listing in computer readable form, which is incorporated herein by reference.

### FIELD OF THE INVENTION

The present invention relates generally to processes for enhancing enzymatic hydrolysis of biomass in a continuous reactor with enzyme compositions comprising a cellulase composition and a xylanase composition in ratios that achieve sugar yields comparable to those achieved in batch only processes. The invention also relates to processes for obtaining hydrolysis products and fermentation products.

### DESCRIPTION OF RELATED ART

Renewable energy sources provide an alternative to current fossil fuel dependence. Production of ethanol as an energy source includes the basic steps of hydrolysis and fermentation. These steps are integrated within larger processes to obtain ethanol from various source materials.

In the hydrolysis step the source material is hydrolyzed to break down cellulose and/or hemicellulose to fermentable sugars. Hydrolysis processes may include batch reactors, continuously operating reactors, semi-batch reactors or semi-continuous reactors, or a combination thereof. Where the hydrolysis process includes use of, e.g., a mixed flow reactor, a fed batch reactor or a continuously stirred tank reactor (CSTR), or series of such reactors, cost savings may be realized, as well as process advantages, such as ease of construction, increased volume production and decreased downtime.

However such systems also provide limitations such as more detailed operations and possible problems arising from inefficient mixing within the reactor, as well as a startup time required to reach a steady state of operation.

Despite the potential limitations arising from selection of a reactor for hydrolysis, it is desired to boost production of fermentable sugars from the hydrolysis, while maintaining low overall expenditures of both time and resources.

While it is known that simply adding more enzyme can often boost overall sugar production, and, correspondingly, fermentation yields, such an approach is not generally desirable in large scale production of ethanol, due to the increased costs of adding additional enzymes, as well as the possible inhibitory effects from accumulation of hydrolysis products, e.g., cellobiose, glucose and xylose.

There is therefore a need in the art for additional processes of hydrolyzing lignocellulosic biomass that improves the production of fermentable sugars and/or fermentation yields. Where continuously operating reactors are used in hydrolysis processes, there is a particular need for such improvement, in order to achieve yields seen in batch processes, without excessive increase in the total amount of enzymes or in enzyme consumption. The present invention provides such processes.

### SUMMARY OF THE INVENTION

Described herein are processes for saccharifying lignocellulosic material in a continuously operating reactor, e.g., continuously stirred tank reactor (CSTR), to improve the yield of the resultant sugars for fermentation. The yield is improved to levels similar to those achieved in a pure batch process, without significant increase of the total enzyme dosage. Also described are processes for producing a fermentation product from the hydrolyzate of such a saccharification process.

The present invention is based on the surprising discovery that saccharification of a pretreated lignocellulosic material including increasing the percentage of a hemicellulolytic composition in an enzyme composition used in saccharification in a continuously operated reactor-containing process without a significant increase in the total amount of enzyme added, increases the yield of glucose and/or xylose in the resultant hydrolyzate, thereby reducing or eliminating the observed gap in performance between a pure batch system and a continuously operated reactor-containing system.

Thus in one aspect, the invention relates to a process of multi-stage saccharification of a lignocellulosic material, the process comprising saccharifying a lignocellulosic material in a continuously operating reactor (e.g., CSTR), with an enzyme composition comprising a cellulolytic composition and a hemicellulolytic composition in a mass ratio of from about 85:15 to about 65:35 (cellulolytic composition:hemicellulolytic composition); continuing saccharification within an additional reactor in series with the first reactor without further addition of enzymes to form a hydrolyzate, wherein the hydrolyzate has a glucose and/or xylose yield that is improved as compared to the yield from a process without such enzyme composition.

In another aspect, the invention relates to process of producing a fermentation product from a lignocellulosic material, the process comprising saccharifying and fermenting the lignocellulosic material, where the saccharifying comprises saccharifying a lignocellulosic material in a continuously operating reactor (e.g., CSTR) with an enzyme composition comprising a cellulolytic composition and a hemicellulolytic composition in a mass ratio of from about 85:15 to about 65:35 (cellulolytic composition:hemicellulolytic composition); and continuing saccharification within an additional reactor in series with the first reactor without further addition of enzymes to form a hydrolyzate, wherein the hydrolyzate has a glucose and/or xylose yield that is improved as compared to the yield from a process without such enzyme composition and fermentation comprises fermenting of the hydrolyzate to produce a fermentation product.

In a further embodiment the invention provides a process of improving the glucose and/or xylose yield of saccharification of a lignocellulosic material in a CSTR process, the process comprising saccharifying a lignocellulosic material in a continuously operating reactor (e.g., CSTR) with an enzyme composition comprising a cellulolytic composition and a hemicellulolytic composition in a mass ratio of from about 85:15 to about 65:35 (cellulolytic composition:hemicellulolytic composition); and continuing saccharification in an additional reactor in series with the first reactor without further addition of enzymes to form a hydrolyzate wherein the hydrolyzate has a glucose yield and/or a xylose yield that is improved as compared to the yields from a process without such enzyme composition.

### Definitions

Acetylxylan esterase: The term “acetylxylan esterase” means a carboxylesterase (EC 3.1.1.72) that catalyzes the hydrolysis of acetyl groups from polymeric xylan, acetylated xylose, acetylated glucose, alpha-napthyl acetate, and p-nitrophenyl acetate. Acetylxylan esterase activity can be determined using 0.5 mM p-nitrophenylacetate as substrate in 50 mM sodium acetate pH 5.0 containing 0.01% TWEEN™ 20 (polyoxyethylene sorbitan monolaurate). One unit of acetylxylan esterase is defined as the amount of enzyme capable of releasing 1 μmole of p-nitrophenolate anion per minute at pH 5, 25° C.

Allelic variant: The term “allelic variant” means any of two or more (e.g., several) alternative forms of a gene occupying the same chromosomal locus. Allelic variation arises naturally through mutation, and may result in polymorphism within populations. Gene mutations can be silent (no change in the encoded polypeptide) or may encode polypeptides having altered amino acid sequences. An allelic variant of a polypeptide is a polypeptide encoded by an allelic variant of a gene.

Alpha-L-arabinofuranosidase: The term “alpha-L-arabinofuranosidase” means an alpha-L-arabinofuranoside arabinofuranohydrolase (EC 3.2.1.55) that catalyzes the hydrolysis of terminal non-reducing alpha-L-arabinofuranoside residues in alpha-L-arabinosides. The enzyme acts on alpha-L-arabinofuranosides, alpha-L-arabinans containing (1,3)- and/or (1,5)-linkages, arabinoxylans, and arabinogalactans. Alpha-L-arabinofuranosidase is also known as arabinosidase, alpha-arabinosidase, alpha-L-arabinosidase, alpha-arabinofuranosidase, polysaccharide alpha-L-arabinofuranosidase, alpha-L-arabinofuranoside hydrolase, L-arabinosidase, or alpha-L-arabinanase. Alpha-L-arabinofuranosidase activity can be determined using 5 mg of medium viscosity wheat arabinoxylan (Megazyme International Ireland, Ltd., Bray, Co. Wicklow, Ireland) per ml of 100 mM sodium acetate pH 5 in a total volume of 200 μl for 30 minutes at 40° C. followed by arabinose analysis by AMINEX® HPX-87H column chromatography (Bio-Rad Laboratories, Inc., Hercules, Calif., USA).

Alpha-glucuronidase: The term “alpha-glucuronidase” means an alpha-D-glucosiduronate glucuronohydrolase (EC 3.2.1.139) that catalyzes the hydrolysis of an alpha-D-glucuronoside to D-glucuronate and an alcohol. Alpha-glucuronidase activity can be determined according to de Vries, 1998, J. Bacteriol. 180: 243-249. One unit of alpha-glucuronidase equals the amount of enzyme capable of releasing 1 μmole of glucuronic or 4-O-methylglucuronic acid per minute at pH 5, 40° C.

Auxiliary Activity 9 polypeptide: The term “Auxiliary Activity 9 polypeptide” or “AA9 polypeptide” means a polypeptide classified as a lytic polysaccharide monooxygenase (Quinlan et al., 2011, Proc. Natl. Acad. Sci. USA 208: 15079-15084; Phillips et al., 2011, ACS Chem. Biol. 6: 1399-1406; Lin et al., 2012, Structure 20: 1051-1061). AA9 polypeptides were formerly classified into the glycoside hydrolase Family 61 (GH61) according to Henrissat, 1991, Biochem. J. 280: 309-316, and Henrissat and Bairoch, 1996, Biochem. J. 316: 695-696.

AA9 polypeptides enhance the hydrolysis of a cellulosic material by an enzyme having cellulolytic activity. Cellulolytic enhancing activity can be determined by measuring the increase in reducing sugars or the increase of the total of cellobiose and glucose from the hydrolysis of a cellulosic material by cellulolytic enzyme under the following conditions: 1-50 mg of total protein/g of cellulose in pretreated corn stover (PCS), wherein total protein is comprised of 50-99.5% w/w cellulolytic enzyme protein and 0.5-50% w/w protein of an AA9 polypeptide for 1-7 days at a suitable temperature, such as 40° C.-80° C., e.g., 40° C., 45° C., 50° C., 55° C., 60° C., 65° C., 70° C., 75° C., or 80° C., and a suitable pH, such as 4-9, e.g., 4.0, 4.5, 5.0, 5.5, 6.0, 6.5, 7.0, 7.5, 8.0, 8.5, or 9.0, compared to a control hydrolysis with equal total protein loading without cellulolytic enhancing activity (1-50 mg of cellulolytic protein/g of cellulose in PCS).

AA9 polypeptide enhancing activity can be determined using a mixture of CELLUCLAST® 1.5L (Novozymes A/S, Bagsværd, Denmark) and beta-glucosidase as the source of the cellulolytic activity, wherein the beta-glucosidase is present at a weight of at least 2-5% protein of the cellulase protein loading. In one aspect, the beta-glucosidase is an Aspergillus oryzae beta-glucosidase (e.g., recombinantly produced in Aspergillus oryzae according to WO 02/095014). In another aspect, the beta-glucosidase is an Aspergillus fumigatus beta-glucosidase (e.g., recombinantly produced in Aspergillus oryzae as described in WO 02/095014).

AA9 polypeptide enhancing activity can also be determined by incubating an AA9 polypeptide with 0.5% phosphoric acid swollen cellulose (PASC), 100 mM sodium acetate pH 5, 1 mM MnSO₄, 0.1% gallic acid, 0.025 mg/ml of Aspergillus fumigatus beta-glucosidase, and 0.01% TRITON® X-100 (4-(1,1,3,3-tetramethylbutyl)phenyl-polyethylene glycol) for 24-96 hours at 40° C. followed by determination of the glucose released from the PASC.

AA9 polypeptide enhancing activity can also be determined according to WO 2013/028928 for high temperature compositions.

AA9 polypeptides enhance the hydrolysis of a cellulosic material catalyzed by enzyme having cellulolytic activity by reducing the amount of cellulolytic enzyme required to reach the same degree of hydrolysis preferably at least 1.01-fold, e.g., at least 1.05-fold, at least 1.10-fold, at least 1.25-fold, at least 1.5-fold, at least 2-fold, at least 3-fold, at least 4-fold, at least 5-fold, at least 10-fold, or at least 20-fold.

The AA9 polypeptide can also be used in the presence of a soluble activating divalent metal cation according to WO 2008/151043 or WO 2012/122518, e.g., copper.

The AA9 polypeptide can be used in the presence of a dioxy compound, a bicylic compound, a heterocyclic compound, a nitrogen-containing compound, a quinone compound, a sulfur-containing compound, or a liquor obtained from a pretreated cellulosic or hemicellulosic material such as pretreated corn stover (WO 2012/021394, WO 2012/021395, WO 2012/021396, WO 2012/021399, WO 2012/021400, WO 2012/021401, WO 2012/021408, and WO 2012/021410).

Beta-glucosidase: The term “beta-glucosidase” means a beta-D-glucoside glucohydrolase (E.C. 3.2.1.21) that catalyzes the hydrolysis of terminal non-reducing beta-D-glucose residues with the release of beta-D-glucose. Beta-glucosidase activity can be determined using p-nitrophenyl-beta-D-glucopyranoside as substrate according to the procedure of Venturi et al., 2002, J. Basic Microbiol. 42: 55-66. One unit of beta-glucosidase is defined as 1.0 μmole of p-nitrophenolate anion produced per minute at 25° C., pH 4.8 from 1 mM p-nitrophenyl-beta-D-glucopyranoside as substrate in 50 mM sodium citrate containing 0.01% TWEEN® 20.

Beta-xylosidase: The term “beta-xylosidase” means a beta-D-xyloside xylohydrolase (E.C. 3.2.1.37) that catalyzes the exo-hydrolysis of short beta (1→4)-xylooligosaccharides to remove successive D-xylose residues from non-reducing termini. Beta-xylosidase activity can be determined as set forth in Example 6 herein.

Catalase: The term “catalase” means a hydrogen-peroxide:hydrogen-peroxide oxidoreductase (EC 1.11.1.6) that catalyzes the conversion of 2H₂O₂ to O₂+2 H₂O. For purposes of the present invention, catalase activity is determined according to U.S. Pat. No. 5,646,025. One unit of catalase activity equals the amount of enzyme that catalyzes the oxidation of 1 μmole of hydrogen peroxide under the assay conditions.

Catalytic domain: The term “catalytic domain” means the region of an enzyme containing the catalytic machinery of the enzyme.

cDNA: The term “cDNA” means a DNA molecule that can be prepared by reverse transcription from a mature, spliced, mRNA molecule obtained from a eukaryotic or prokaryotic cell. cDNA lacks intron sequences that may be present in the corresponding genomic DNA. The initial, primary RNA transcript is a precursor to mRNA that is processed through a series of steps, including splicing, before appearing as mature spliced mRNA.

Cellobiohydrolase: The term “cellobiohydrolase” means a 1,4-beta-D-glucan cellobiohydrolase (E.C. 3.2.1.91 and E.C. 3.2.1.176) that catalyzes the hydrolysis of 1,4-beta-D-glucosidic linkages in cellulose, cellooligosaccharides, or any beta-1,4-linked glucose containing polymer, releasing cellobiose from the reducing end (cellobiohydrolase I) or non-reducing end (cellobiohydrolase II) of the chain (Teeri, 1997, Trends in Biotechnology 15: 160-167; Teeri et al., 1998, Biochem. Soc. Trans. 26: 173-178). Cellobiohydrolase activity can be determined according to the procedures described by Lever et al., 1972, Anal. Biochem. 47: 273-279; van Tilbeurgh et al., 1982, FEBS Letters 149: 152-156; van Tilbeurgh and Claeyssens, 1985, FEBS Letters 187: 283-288; and Tomme et al., 1988, Eur. J. Biochem. 170: 575-581.

Cellulolytic enzyme, cellulolytic composition, or cellulase: The term “cellulolytic enzyme,” “cellulolytic composition”, “cellulolytic composition”, or “cellulase” means one or more (e.g., several) enzymes that hydrolyze a cellulosic material. Such enzymes include endoglucanase(s), cellobiohydrolase(s), beta-glucosidase(s), or combinations thereof. The two basic approaches for measuring cellulolytic enzyme activity include: (1) measuring the total cellulolytic enzyme activity, and (2) measuring the individual cellulolytic enzyme activities (endoglucanases, cellobiohydrolases, and beta-glucosidases) as reviewed in Zhang et al., 2006, Biotechnology Advances 24: 452-481. Total cellulolytic enzyme activity can be measured using insoluble substrates, including Whatman No 1 filter paper, microcrystalline cellulose, bacterial cellulose, algal cellulose, cotton, pretreated lignocellulose, etc. The most common total cellulolytic activity assay is the filter paper assay using Whatman No 1 filter paper as the substrate. The assay was established by the International Union of Pure and Applied Chemistry (IUPAC) (Ghose, 1987, Pure Appl. Chem. 59: 257-68). Cellulase activity can be determined as set forth in Example 4 herein.

Cellulolytic enzyme activity can be determined by measuring the increase in production/release of sugars during hydrolysis of a cellulosic material by cellulolytic enzyme(s) under the following conditions: 1-50 mg of cellulolytic enzyme protein/g of cellulose in pretreated corn stover (PCS) (or other pretreated cellulosic material) for 3-7 days at a suitable temperature such as 40° C.-80° C., e.g., 50° C., 55° C., 60° C., 65° C., or 70° C., and a suitable pH such as 4-9, e.g., 5.0, 5.5, 6.0, 6.5, or 7.0, compared to a control hydrolysis without addition of cellulolytic enzyme protein. Typical conditions are 1 ml reactions, washed or unwashed PCS, 5% insoluble solids (dry weight), 50 mM sodium acetate pH 5, 1 mM MnSO₄, 50° C., 55° C., or 60° C., 72 hours, sugar analysis by an AMINEX® HPX-87H column (Bio-Rad Laboratories, Inc., Hercules, Calif., USA).

Coding sequence: The term “coding sequence” means a polynucleotide, which directly specifies the amino acid sequence of a polypeptide. The boundaries of the coding sequence are generally determined by an open reading frame, which begins with a start codon such as ATG, GTG, or TTG and ends with a stop codon such as TAA, TAG, or TGA. The coding sequence may be a genomic DNA, cDNA, synthetic DNA, or a combination thereof.

Control sequences: The term “control sequences” means nucleic acid sequences necessary for expression of a polynucleotide encoding a mature polypeptide of the present invention. Each control sequence may be native (i.e., from the same gene) or foreign (i.e., from a different gene) to the polynucleotide encoding the polypeptide or native or foreign to each other. Such control sequences include, but are not limited to, a leader, polyadenylation sequence, propeptide sequence, promoter, signal peptide sequence, and transcription terminator. At a minimum, the control sequences include a promoter, and transcriptional and translational stop signals. The control sequences may be provided with linkers for the purpose of introducing specific restriction sites facilitating ligation of the control sequences with the coding region of the polynucleotide encoding a polypeptide.

Endoglucanase: The term “endoglucanase” means a 4-(1,3;1,4)-beta-D-glucan 4-glucanohydrolase (E.C. 3.2.1.4) that catalyzes endohydrolysis of 1,4-beta-D-glycosidic linkages in cellulose, cellulose derivatives (such as carboxymethyl cellulose and hydroxyethyl cellulose), lichenin, beta-1,4 bonds in mixed beta-1,3-1,4 glucans such as cereal beta-D-glucans or xyloglucans, and other plant material containing cellulosic components. Endoglucanase activity can be determined by measuring reduction in substrate viscosity or increase in reducing ends determined by a reducing sugar assay (Zhang et al., 2006, supra). Endoglucanase activity can also be determined using carboxymethyl cellulose (CMC) as substrate according to the procedure of Ghose, 1987, supra, at pH 5, 40° C.

Expression: The term “expression” includes any step involved in the production of a polypeptide including, but not limited to, transcription, post-transcriptional modification, translation, post-translational modification, and secretion.

Expression vector: The term “expression vector” means a linear or circular DNA molecule that comprises a polynucleotide encoding a polypeptide and is operably linked to control sequences that provide for its expression.

Feruloyl esterase: The term “feruloyl esterase” means a 4-hydroxy-3-methoxycinnamoyl-sugar hydrolase (EC 3.1.1.73) that catalyzes the hydrolysis of 4-hydroxy-3-methoxycinnamoyl (feruloyl) groups from esterified sugar, which is usually arabinose in natural biomass substrates, to produce ferulate (4-hydroxy-3-methoxycinnamate). Feruloyl esterase (FAE) is also known as ferulic acid esterase, hydroxycinnamoyl esterase, FAE-III, cinnamoyl ester hydrolase, FAEA, cinnAE, FAE-I, or FAE-II. Feruloyl esterase activity can be determined using 0.5 mM p-nitrophenylferulate as substrate in 50 mM sodium acetate pH 5.0. One unit of feruloyl esterase equals the amount of enzyme capable of releasing 1 μmole of p-nitrophenolate anion per minute at pH 5, 25° C.

Fragment: The term “fragment” means a polypeptide having one or more (e.g., several) amino acids absent from the amino and/or carboxyl terminus of a mature polypeptide main; wherein the fragment has enzyme activity. In one aspect, a fragment contains at least 85%, e.g., at least 90% or at least 95% of the amino acid residues of the mature polypeptide of an enzyme.

High stringency conditions: The term “high stringency conditions” means for probes of at least 100 nucleotides in length, prehybridization and hybridization at 42° C. in 5×SSPE, 0.3% SDS, 200 micrograms/ml sheared and denatured salmon sperm DNA, and 50% formamide, following standard Southern blotting procedures for 12 to 24 hours. The carrier material is finally washed three times each for 15 minutes using 0.2×SSC, 0.2% SDS at 65° C.

Hemicellulolytic enzyme, hemicellulolytic composition or hemicellulase: The term “hemicellulolytic enzyme”, “hemicellulolytic composition,” “hemicellulolytic composition” or “hemicellulase” means one or more (e.g., several) enzymes that hydrolyze a hemicellulosic material. See, for example, Shallom and Shoham, 2003, Current Opinion In Microbiology 6(3): 219-228). Hemicellulases are key components in the degradation of plant biomass. Examples of hemicellulases include, but are not limited to, an acetylmannan esterase, an acetylxylan esterase, an arabinanase, an arabinofuranosidase, a coumaric acid esterase, a feruloyl esterase, a galactosidase, a glucuronidase, a glucuronoyl esterase, a mannanase, a mannosidase, a xylanase, and a xylosidase. The substrates for these enzymes, hemicelluloses, are a heterogeneous group of branched and linear polysaccharides that are bound via hydrogen bonds to the cellulose microfibrils in the plant cell wall, crosslinking them into a robust network. Hemicelluloses are also covalently attached to lignin, forming together with cellulose a highly complex structure. The variable structure and organization of hemicelluloses require the concerted action of many enzymes for its complete degradation. The catalytic modules of hemicellulases are either glycoside hydrolases (GHs) that hydrolyze glycosidic bonds, or carbohydrate esterases (CEs), which hydrolyze ester linkages of acetate or ferulic acid side groups. These catalytic modules, based on homology of their primary sequence, can be assigned into GH and CE families. Some families, with an overall similar fold, can be further grouped into clans, marked alphabetically (e.g., GH-A). A most informative and updated classification of these and other carbohydrate active enzymes is available in the Carbohydrate-Active Enzymes (CAZy) database. Hemicellulolytic enzyme activities can be measured according to Ghose and Bisaria, 1987, Pure & Appl. Chem. 59: 1739-1752, at a suitable temperature such as 40° C.-80° C., e.g., 50° C., 55° C., 60° C., 65° C., or 70° C., and a suitable pH such as 4-9, e.g., 5.0, 5.5, 6.0, 6.5, or 7.0.

Homologous 3′ or 5′ region: The term “homologous 3′ region” means a fragment of DNA that is identical in sequence or has a sequence identity of at least 70%, e.g., at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% to a region in the genome and when combined with a homologous 5′ region can target integration of a piece of DNA to a specific site in the genome by homologous recombination. The term “homologous 5′ region” means a fragment of DNA that is identical in sequence to a region in the genome and when combined with a homologous 3′ region can target integration of a piece of DNA to a specific site in the genome by homologous recombination. The homologous 5′ and 3′ regions must be linked in the genome which means they are on the same chromosome and within at least 200 kb of one another.

Homologous flanking region: The term “homologous flanking region” means a fragment of DNA that is identical or has a sequence identity of at least 70%, e.g., at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% to a region in the genome and is located immediately upstream or downstream of a specific site in the genome into which extracellular DNA is targeted for integration.

Homologous repeat: The term “homologous repeat” means a fragment of DNA that is repeated at least twice in the recombinant DNA introduced into a host cell and which can facilitate the loss of the DNA, i.e., selectable marker that is inserted between two homologous repeats, by homologous recombination. A homologous repeat is also known as a direct repeat.

Host cell: The term “host cell” means any cell type that is susceptible to transformation, transfection, transduction, or the like with a nucleic acid construct or expression vector comprising a polynucleotide encoding a polypeptide. The term “host cell” encompasses any progeny of a parent cell that is not identical to the parent cell due to mutations that occur during replication.

Isolated: The term “isolated” means a substance in a form or environment that does not occur in nature. Non-limiting examples of isolated substances include (1) any non-naturally occurring substance, (2) any substance including, but not limited to, any enzyme, variant, nucleic acid, protein, peptide or cofactor, that is at least partially removed from one or more or all of the naturally occurring constituents with which it is associated in nature; (3) any substance modified by the hand of man relative to that substance found in nature; or (4) any substance modified by increasing the amount of the substance relative to other components with which it is naturally associated (e.g., recombinant production in a host cell; multiple copies of a gene encoding the substance; and use of a stronger promoter than the promoter naturally associated with the gene encoding the substance).

Low stringency conditions: The term “low stringency conditions” means for probes of at least 100 nucleotides in length, prehybridization and hybridization at 42° C. in 5×SSPE, 0.3% SDS, 200 micrograms/ml sheared and denatured salmon sperm DNA, and 25% formamide, following standard Southern blotting procedures for 12 to 24 hours. The carrier material is finally washed three times each for 15 minutes using 0.2×SSC, 0.2% SDS at 50° C.

Mature polypeptide: The term “mature polypeptide” means a polypeptide in its final form following translation and any post-translational modifications, such as N-terminal processing, C-terminal truncation, glycosylation, phosphorylation, etc. For instance, the mature polypeptide may be identified, using, e.g., the SignalP program (Nielsen et al., 1997, Protein Engineering 10: 1-6) that predicts a portion of the amino acid sequence as a signal peptide. As such, the mature polypeptide would be identified as the sequence lacking such redicted signal portion.

In one aspect, the mature polypeptide of a beta-glucosidase is amino acids 20 to 863 of SEQ ID NO: 2 based on the SignalP 3.0 program (Bendtsen et al., 2004, J. Mol. Biol. 340: 783-795) that predicts amino acids 1 to 19 of SEQ ID NO: 2 are a signal peptide. In another aspect, the mature polypeptide of a beta-glucosidase variant is amino acids 20 to 863 of SEQ ID NO: 4 based on the SignalP 3.0 program that predicts amino acids 1 to 19 of SEQ ID NO: 4 are a signal peptide. In another aspect, the mature polypeptide of a cellobiohydrolase I is amino acids 27 to 532 of SEQ ID NO: 6 based on the SignalP 3.0 program that predicts amino acids 1 to 26 of SEQ ID NO: 6 are a signal peptide. In another aspect, the mature polypeptide of a cellobiohydrolase 11 is amino acids 20 to 454 of SEQ ID NO: 8 based on the SignalP 3.0 program that predicts amino acids 1 to 19 of SEQ ID NO: 8 are a signal peptide. In another aspect, the mature polypeptide of In another aspect, the mature polypeptide of an AA9 polypeptide is amino acids 26 to 253 of SEQ ID NO: 10 based on the SignalP 3.0 program that predicts amino acids 1 to 25 of SEQ ID NO: 10 are a signal peptide. In another aspect, the mature polypeptide of a GH10 xylanase is amino acids 20 to 397 of SEQ ID NO: 12 based on the SignalP 3.0 program that predicts amino acids 1 to 19 of SEQ ID NO: 12 are a signal peptide. In another aspect, the mature polypeptide of a beta-xylosidase is amino acids 21 to 792 of SEQ ID NO: 14 based on the SignalP 3.0 program that predicts amino acids 1 to 20 of SEQ ID NO: 14 are a signal peptide.

It is known in the art that a host cell may produce a mixture of two of more different mature polypeptides (i.e., with a different C-terminal and/or N-terminal amino acid) expressed by the same polynucleotide.

Mature polypeptide coding sequence: The term “mature polypeptide coding sequence” means a polynucleotide that encodes a mature polypeptide having enzyme activity.

Medium stringency conditions: The term “medium stringency conditions” means for probes of at least 100 nucleotides in length, prehybridization and hybridization at 42° C. in 5×SSPE, 0.3% SDS, 200 micrograms/ml sheared and denatured salmon sperm DNA, and 35% formamide, following standard Southern blotting procedures for 12 to 24 hours. The carrier material is finally washed three times each for 15 minutes using 0.2×SSC, 0.2% SDS at 55° C.

Medium-high stringency conditions: The term “medium-high stringency conditions” means for probes of at least 100 nucleotides in length, prehybridization and hybridization at 42° C. in 5×SSPE, 0.3% SDS, 200 micrograms/ml sheared and denatured salmon sperm DNA, and 35% formamide, following standard Southern blotting procedures for 12 to 24 hours. The carrier material is finally washed three times each for 15 minutes using 0.2×SSC, 0.2% SDS at 60° C.

Nucleic acid construct: The term “nucleic acid construct” means a nucleic acid molecule, either single- or double-stranded, which is isolated from a naturally occurring gene or is modified to contain segments of nucleic acids in a manner that would not otherwise exist in nature or which is synthetic, which comprises one or more control sequences.

Operably linked: The term “operably linked” means a configuration in which a control sequence is placed at an appropriate position relative to the coding sequence of a polynucleotide such that the control sequence directs expression of the coding sequence.

Parent Enzyme: The term “parent” means an enzyme to which an alteration is made to produce a variant. The parent may be a naturally occurring (wild-type) polypeptide or a variant thereof.

Pretreated cellulosic or hemicellulosic material: The term “pretreated cellulosic or hemicellulosic material” means a cellulosic or hemicellulosic material derived from biomass by treatment with heat and dilute sulfuric acid, alkaline pretreatment, neutral pretreatment, or any pretreatment known in the art.

Pretreated corn stover: The term “Pretreated Corn Stover” or “PCS” means a cellulosic material derived from corn stover by treatment with heat and dilute sulfuric acid, alkaline pretreatment, neutral pretreatment, or any pretreatment known in the art.

Sequence identity: The relatedness between two amino acid sequences or between two nucleotide sequences is described by the parameter “sequence identity”.

For purposes of the present invention, the sequence identity between two amino acid sequences is determined using the Needleman-Wunsch algorithm (Needleman and Wunsch, 1970, J. Mol. Biol. 48: 443-453) as implemented in the Needle program of the EMBOSS package (EMBOSS: The European Molecular Biology Open Software Suite, Rice et al., 2000, Trends Genet. 16: 276-277), preferably version 5.0.0 or later. The parameters used are gap open penalty of 10, gap extension penalty of 0.5, and the EBLOSUM62 (EMBOSS version of BLOSUM62) substitution matrix. The output of Needle labeled “longest identity” (obtained using the -nobrief option) is used as the percent identity and is calculated as follows:

(Identical Residues×100)/(Length of Alignment−Total Number of Gaps in Alignment)

For purposes of the present invention, the sequence identity between two deoxyribonucleotide sequences is determined using the Needleman-Wunsch algorithm (Needleman and Wunsch, 1970, supra) as implemented in the Needle program of the EMBOSS package (EMBOSS: The European Molecular Biology Open Software Suite, Rice et al., 2000, supra), preferably version 5.0.0 or later. The parameters used are gap open penalty of 10, gap extension penalty of 0.5, and the EDNAFULL (EMBOSS version of NCBI NUC4.4) substitution matrix. The output of Needle labeled “longest identity” (obtained using the -nobrief option) is used as the percent identity and is calculated as follows:

(Identical Deoxyribonucleotides×100)/(Length of Alignment−Total Number of Gaps in Alignment)

Subsequence: The term “subsequence” means a polynucleotide having one or more (e.g., several) nucleotides absent from the 5′ and/or 3′ end of a mature polypeptide coding sequence, wherein the subsequence encodes a fragment having enzyme activity. In one aspect, a subsequence contains at least 85%, e.g., at least 90% or at least 95% of the nucleotides of the mature polypeptide coding sequence of an enzyme.

Transformant: The term “transformant” means a cell which has taken up extracellular DNA (foreign, artificial or modified) and expresses the gene(s) contained therein.

Transformation: The term “transformation” means the introduction of extracellular DNA into a cell, i.e., the genetic alteration of a cell resulting from the direct uptake, incorporation and expression of exogenous genetic material (exogenous DNA) from its surroundings and taken up through the cell membrane(s).

Variant: The term “variant” means a polypeptide having enzyme or enzyme enhancing activity comprising an alteration, i.e., a substitution, insertion, and/or deletion, at one or more (e.g., several) positions. A substitution means replacement of the amino acid occupying a position with a different amino acid; a deletion means removal of the amino acid occupying a position; and an insertion means adding an amino acid adjacent to and immediately following the amino acid occupying a position.

Very high stringency conditions: The term “very high stringency conditions” means for probes of at least 100 nucleotides in length, prehybridization and hybridization at 42° C. in 5×SSPE, 0.3% SDS, 200 micrograms/ml sheared and denatured salmon sperm DNA, and 50% formamide, following standard Southern blotting procedures for 12 to 24 hours. The carrier material is finally washed three times each for 15 minutes using 0.2×SSC, 0.2% SDS at 70° C.

Very low stringency conditions: The term “very low stringency conditions” means for probes of at least 100 nucleotides in length, prehybridization and hybridization at 42° C. in 5×SSPE, 0.3% SDS, 200 micrograms/ml sheared and denatured salmon sperm DNA, and 25% formamide, following standard Southern blotting procedures for 12 to 24 hours. The carrier material is finally washed three times each for 15 minutes using 0.2×SSC, 0.2% SDS at 45° C.

Whole broth preparation: The term “whole broth preparation” means a composition produced by a naturally-occurring source, i.e., a naturally-occurring microorganism that is unmodified with respect to the cellulolytic and/or hemicellulolytic enzymes produced by the naturally-occurring microorganism, or a non-naturally-occurring source, i.e., a non-naturally-occurring microorganism, e.g., mutant, that is unmodified with respect to the cellulolytic and/or hemicellulolytic enzymes produced by the non-naturally-occurring microorganism.

Wild-Type Enzyme: The term “wild-type” enzyme means an enzyme expressed by a naturally occurring microorganism, such as a bacterium, yeast, or filamentous fungus found in nature.

Xylan-containing material: The term “xylan-containing material” means any material comprising a plant cell wall polysaccharide containing a backbone of beta-(1-4)-linked xylose residues. Xylans of terrestrial plants are heteropolymers possessing a beta-(1-4)-D-xylopyranose backbone, which is branched by short carbohydrate chains. They comprise D-glucuronic acid or its 4-O-methyl ether, L-arabinose, and/or various oligosaccharides, composed of D-xylose, L-arabinose, D- or L-galactose, and D-glucose. Xylan-type polysaccharides can be divided into homoxylans and heteroxylans, which include glucuronoxylans, (arabino)glucuronoxylans, (glucurono)arabinoxylans, arabinoxylans, and complex heteroxylans. See, for example, Ebringerova et al., 2005, Adv. Polym. Sci. 186: 1-67.

In processes of the present invention, any material containing xylan may be used. In a preferred aspect, the xylan-containing material is lignocellulose.

Xylan degrading activity or xylanolytic activity: The term “xylan degrading activity” or “xylanolytic activity” means a biological activity that hydrolyzes xylan-containing material. The two basic approaches for measuring xylanolytic activity include: (1) measuring the total xylanolytic activity, and (2) measuring the individual xylanolytic activities (e.g., endoxylanases, beta-xylosidases, arabinofuranosidases, alpha-glucuronidases, acetylxylan esterases, feruloyl esterases, and alpha-glucuronyl esterases). Recent progress in assays of xylanolytic enzymes was summarized in several publications including Biely and Puchard, 2006, Journal of the Science of Food and Agriculture 86(11): 1636-1647; Spanikova and Biely, 2006, FEBS Letters 580(19): 4597-4601; Herrmann et al., 1997, Biochemical Journal 321: 375-381.

Total xylan degrading activity can be measured by determining the reducing sugars formed from various types of xylan, including, for example, oat spelt, beechwood, and larchwood xylans, or by photometric determination of dyed xylan fragments released from various covalently dyed xylans. A common total xylanolytic activity assay is based on production of reducing sugars from polymeric 4-O-methyl glucuronoxylan as described in Bailey et al., 1992, Interlaboratory testing of methods for assay of xylanase activity, Journal of Biotechnology 23(3): 257-270. Xylanase activity can also be determined with 0.2% AZCL-arabinoxylan as substrate in 0.01% TRITON® X-100 and 200 mM sodium phosphate pH 6 at 37° C. One unit of xylanase activity is defined as 1.0 μmole of azurine produced per minute at 37° C., pH 6 from 0.2% AZCL-arabinoxylan as substrate in 200 mM sodium phosphate pH 6.

Xylan degrading activity can be determined by measuring the increase in hydrolysis of birchwood xylan (Sigma Chemical Co., Inc., St. Louis, Mo., USA) by xylan-degrading enzyme(s) under the following typical conditions: 1 ml reactions, 5 mg/ml substrate (total solids), 5 mg of xylanolytic protein/g of substrate, 50 mM sodium acetate pH 5, 50° C., 24 hours, sugar analysis using p-hydroxybenzoic acid hydrazide (PHBAH) assay as described by Lever, 1972, Anal. Biochem. 47: 273-279.

Xylanase: The term “xylanase” means a 1,4-beta-D-xylan-xylohydrolase (E.C. 3.2.1.8) that catalyzes the endohydrolysis of 1,4-beta-D-xylosidic linkages in xylans. Xylanase activity can be determined as set forth in Example 5 herein.

Reference to “about” a value or parameter herein includes aspects that are directed to that value or parameter per se. For example, description referring to “about X” includes the aspect “X”.

As used herein and in the appended claims, the singular forms “a,” “or,” and “the” include plural referents unless the context clearly dictates otherwise. It is understood that the aspects of the invention described herein include “consisting” and/or “consisting essentially of” aspects.

Unless defined otherwise or clearly indicated by context, all technical and scientific terms used herein have the same meaning as commonly understood by one of ordinary skill in the art to which this invention belongs.

### BRIEF DESCRIPTION OF THE DRAWINGS

FIG. 1 is a graph showing the glucose yield of two hydrolysis samples as described in Example 1.

FIG. 2 provides graphs of the sugar yield for pretreated wheat straw and arundo, as described in Example 2, where FIG. 2A shows the final glucose concentrations of the supernatant samples and FIG. 2B shows the final glucose concentrations of the supernatant samples.

FIG. 3 provides a graph of the glucose yield from two hydrolysis processes, illustrating closure of the gap attributable to a CSTR-containing process, as described in Example 3.

### DETAILED DESCRIPTION

Described herein are processes for improving the sugar yield from a multi stage hydrolysis comprising use of a continuously operating reactor (e.g., CSTR). Further described are processes of hydrolysis and processes of fermentation incorporating such improved sugar yield processes. Also described are enzyme compositions suitable for use in the processes and/or methods described herein.

The present inventors have surprisingly found that by contacting a lignocellulose-containing material with an enzyme composition comprising both a cellulolytic composition and a hemicellulolytic composition in a specified ratio, that a lag in performance previously observed in a multi stage saccharification process including a continuously operating reactor, as compared to a pure batch process, can be improved or overcome.

As used herein, the term “improved” with respect to an improved sugar yield refers to achieving an increased yield, as compared to the yield obtained from an equivalent process not utilizing enzyme compositions as described herein.

The enzyme compositions used in processes of the invention comprise varied percentages of a hemicellulolytic composition without a significant increase in the total enzyme dosage. It was found that use of an enzyme composition comprising at least about 15% of a hemicellulolytic composition as described herein resulted in improved sugar concentrations, as compared to saccharification in a multi stage saccharification process including the same reactor setup, but without use of such enzyme compositions, e.g., enzyme compositions comprising 0% to less than about 15% of a hemicellulolytic composition. It was correspondingly determined that use of an enzyme composition comprising at least about 12.75 U of xylanase per g of cellulosic material and at least about 0.15 U of beta-xylosidase per g of cellulosic material as described herein resulted in improved sugar concentrations, as compared to saccharification in a multi stage saccharification process including the same reactor setup, but without use of such enzyme compositions.

### Cellulosic Material

Processes of the present invention are carried out using cellulosic material. The term “cellulosic material” means any material containing cellulose. The predominant polysaccharide in the primary cell wall of biomass is cellulose, the second most abundant is hemicellulose, and the third is pectin. The secondary cell wall, produced after the cell has stopped growing, also contains polysaccharides and is strengthened by polymeric lignin covalently cross-linked to hemicellulose. Cellulose is a homopolymer of anhydrocellobiose and thus a linear beta-(1-4)-D-glucan, while hemicelluloses include a variety of compounds, such as xylans, xyloglucans, arabinoxylans, and mannans in complex branched structures with a spectrum of substituents. Although generally polymorphous, cellulose is found in plant tissue primarily as an insoluble crystalline matrix of parallel glucan chains. Hemicelluloses usually hydrogen bond to cellulose, as well as to other hemicelluloses, which help stabilize the cell wall matrix.

Cellulose is generally found, for example, in the stems, leaves, hulls, husks, and cobs of plants or leaves, branches, and wood of trees. The cellulosic material may be, but is not limited to, agricultural residue, herbaceous material (including energy crops), municipal solid waste, pulp and paper mill residue, waste paper, and wood (including forestry residue) (see, for example, Wiselogel et al., 1995, in Handbook on Bioethanol (Charles E. Wyman, editor), pp. 105-118, Taylor & Francis, Washington D.C.; Wyman, 1994, Bioresource Technology 50: 3-16; Lynd, 1990, Applied Biochemistry and Biotechnology 24/25: 695-719; Mosier et al., 1999, Recent Progress in Bioconversion of Lignocellulosics, in Advances in Biochemical Engineering/Biotechnology, T. Scheper, managing editor, Volume 65, pp. 23-40, Springer-Verlag, New York). It is understood herein that the cellulose may be in the form of lignocellulose, a plant cell wall material containing lignin, cellulose, and hemicellulose in a mixed matrix. In a preferred embodiment, the cellulosic material is any biomass material. In another preferred embodiment the cellulosic material is lignocellulose-containing biomass material. In another preferred embodiment, the cellulosic material is lignocellulose, which comprises cellulose, hemicelluloses, and lignin.

In an embodiment, the cellulosic material is agricultural residue, herbaceous material (including energy crops), municipal solid waste, pulp and paper mill residue, waste paper, or wood (including forestry residue).

In another embodiment, the cellulosic material is arundo, bagasse, bamboo, corn cob, corn fiber, corn stover, miscanthus, rice straw, switchgrass, or wheat straw.

In one embodiment, the cellulosic material is fiber, such as corn fiber or wheat fiber. Fiber, such as corn or wheat fiber, may be obtained by fractionation. Fractionation technologies are well-known in the art. In one embodiment the cellulosic material is fiber obtained from dry fractionation processes. In one embodiment the cellulosic material is fiber obtained from wet fractionation processes.

In another embodiment, the cellulosic material is aspen, eucalyptus, fir, pine, poplar, spruce, or willow.

In another embodiment, the cellulosic material is algal cellulose, bacterial cellulose, cotton linter, filter paper, microcrystalline cellulose (e.g., AVICEL®), or phosphoric-acid treated cellulose.

In another embodiment, the cellulosic material is an aquatic biomass. As used herein the term “aquatic biomass” means biomass produced in an aquatic environment by a photosynthesis process. The aquatic biomass may be algae, emergent plants, floating-leaf plants, or submerged plants.

The cellulosic material may be used as is or may be subjected to pretreatment, using conventional methods known in the art, as described more fully herein. In a preferred embodiment, the cellulosic material is pretreated.

### Hemicellulosic Material

The term “hemicellulosic material” means any material comprising hemicelluloses. Hemicelluloses include xylan, glucuronoxylan, arabinoxylan, glucomannan, and xyloglucan. These polysaccharides contain many different sugar monomers. Sugar monomers in hemicellulose can include xylose, mannose, galactose, rhamnose, and arabinose. Hemicelluloses contain most of the D-pentose sugars. Xylose is in most cases the sugar monomer present in the largest amount, although in softwoods mannose can be the most abundant sugar. Xylan contains a backbone of beta-(1-4)-linked xylose residues. Xylans of terrestrial plants are heteropolymers possessing a beta-(1-4)-D-xylopyranose backbone, which is branched by short carbohydrate chains. They comprise D-glucuronic acid or its 4-O-methyl ether, L-arabinose, and/or various oligosaccharides, composed of D-xylose, L-arabinose, D- or L-galactose, and D-glucose. Xylan-type polysaccharides can be divided into homoxylans and heteroxylans, which include glucuronoxylans, (arabino)glucuronoxylans, (glucurono)arabinoxylans, arabinoxylans, and complex heteroxylans. See, for example, Ebringerova et al., 2005, Adv. Polym. Sci. 186: 1-67. Hemicellulosic material is also known herein as “xylan-containing material”.

Sources for hemicellulosic material are essentially the same as those for cellulosic material described herein. It is understood herein that the hemicellulose may be in the form of lignocellulose, a plant cell wall material containing lignin, cellulose, and hemicellulose in a mixed matrix. In a preferred embodiment, the hemicellulosic material is any biomass material. In another preferred embodiment, the hemicellulosic material is lignocellulose, which comprises cellulose, hemicelluloses, and lignin.

### Pretreatment of Cellulosic Material

In practicing the processes of the present invention, the cellulosic material used may be pretreated by any pretreatment process known in the art, used to disrupt plant cell wall components of cellulosic or hemicellulosic material (Chandra et al., 2007, Adv. Biochem. Engin./Biotechnol. 108: 67-93; Galbe and Zacchi, 2007, Adv. Biochem. Engin./Biotechnol. 108: 41-65; Hendriks and Zeeman, 2009, Bioresource Technology 100: 10-18; Mosier et al., 2005, Bioresource Technology 96: 673-686; Taherzadeh and Karimi, 2008, Int. J. Mol. Sci. 9: 1621-1651; Yang and Wyman, 2008, Biofuels Bioproducts and Biorefining-Biofpr. 2: 26-40).

The cellulosic or hemicellulosic material may also be subjected to particle size reduction, sieving, pre-soaking, wetting, washing, and/or conditioning prior to or with additional pretreatment methods, using methods known in the art or as otherwise described herein.

Conventional pretreatments include, but are not limited to, steam pretreatment (with or without explosion), dilute acid pretreatment, hot water pretreatment, alkaline pretreatment, lime pretreatment, wet oxidation, wet explosion, ammonia fiber explosion, organosolv pretreatment, and biological pretreatment. Additional pretreatments include ammonia percolation, ultrasound, electroporation, microwave, supercritical CO₂, supercritical H₂O, ozone, ionic liquid, and gamma irradiation pretreatments.

In an embodiment the cellulosic or hemicellulosic material is pretreated before hydrolysis and/or fermentation. Pretreatment is preferably performed prior to the hydrolysis. Alternatively, the pretreatment can be carried out simultaneously with enzyme hydrolysis to release fermentable sugars, such as glucose, xylose, and/or cellobiose. In most cases the pretreatment step itself results in some conversion of biomass to fermentable sugars (even in absence of enzymes).

Steam Pretreatment. In steam pretreatment, the cellulosic or hemicellulosic material is heated to disrupt the plant cell wall components, including lignin, hemicellulose, and cellulose to make the cellulose and other fractions, e.g., hemicellulose, accessible to enzymes. The cellulosic material is passed to or through a reaction vessel where steam is injected to increase the temperature to the required temperature and pressure and is retained therein for the desired reaction time. Steam pretreatment is preferably performed at 140-250° C., e.g., 160-200° C. or 170-190° C., where the optimal temperature range depends on optional addition of a chemical catalyst. Residence time for the steam pretreatment is preferably 1-60 minutes, e.g., 1-30 minutes, 1-20 minutes, 3-12 minutes, or 4-10 minutes, where the optimal residence time depends on the temperature and optional addition of a chemical catalyst. Steam pretreatment allows for relatively high solids loadings, so that the cellulosic material is generally only moist during the pretreatment. The steam pretreatment is often combined with an explosive discharge of the material after the pretreatment, which is known as steam explosion, that is, rapid flashing to atmospheric pressure and turbulent flow of the material to increase the accessible surface area by fragmentation (Duff and Murray, 1996, Bioresource Technology 855: 1-33; Galbe and Zacchi, 2002, Appl. Microbiol. Biotechnol. 59: 618-628; U.S. Patent Application No. 2002/0164730). During steam pretreatment, hemicellulose acetyl groups are cleaved and the resulting acid autocatalyzes partial hydrolysis of the hemicellulose to monosaccharides and oligosaccharides. Lignin is removed to only a limited extent.

Chemical Pretreatment: The term “chemical pretreatment” refers to any chemical pretreatment that promotes the separation and/or release of cellulose, hemicellulose, and/or lignin. Such a pretreatment may convert crystalline cellulose to amorphous cellulose. Examples of suitable chemical pretreatment processes include, for example, dilute acid pretreatment, lime pretreatment, wet oxidation, ammonia fiber/freeze expansion (AFEX), ammonia percolation (APR), ionic liquid, and organosolv pretreatments.

A chemical catalyst such as H₂SO₄ or SO₂ (typically 0.3 to 5% w/w) is sometimes added prior to steam pretreatment, which decreases the time and temperature, increases the recovery, and improves enzymatic hydrolysis (Ballesteros et al., 2006, Appl. Biochem. Biotechnol. 129-132: 496-508; Varga et al., 2004, Appl. Biochem. Biotechnol. 113-116: 509-523; Sassner et al., 2006, Enzyme Microb. Technol. 39: 756-762). In dilute acid pretreatment, the cellulosic material is mixed with dilute acid, typically H₂SO₄, and water to form a slurry, heated by steam to the desired temperature, and after a residence time flashed to atmospheric pressure. The dilute acid pretreatment may be performed with a number of reactor designs, e.g., plug-flow reactors, counter-current reactors, or continuous counter-current shrinking bed reactors (Duff and Murray, 1996, supra; Schell et al., 2004, Bioresource Technology 91: 179-188; Lee et al., 1999, Adv. Biochem. Eng. Biotechnol. 65: 93-115).

Several methods of pretreatment under alkaline conditions may also be used. These alkaline pretreatments include, but are not limited to, sodium hydroxide, lime, wet oxidation, ammonia percolation (APR), and ammonia fiber/freeze expansion (AFEX) pretreatment.

Lime pretreatment is performed with calcium oxide or calcium hydroxide at temperatures of 85-150° C. and residence times from 1 hour to several days (Wyman et al., 2005, Bioresource Technology 96: 1959-1966; Mosier et al., 2005, supra). WO 2006/110891, WO 2006/110899, WO 2006/110900, and WO 2006/110901 disclose pretreatment methods using ammonia.

Wet oxidation is a thermal pretreatment performed typically at 180-200° C. for 5-15 minutes with addition of an oxidative agent such as hydrogen peroxide or over-pressure of oxygen (Schmidt and Thomsen, 1998, Bioresource Technology 64: 139-151; Palonen et al., 2004, Appl. Biochem. Biotechnol. 117: 1-17; Varga et al., 2004, Biotechnol. Bioeng. 88: 567-574; Martin et al., 2006, J. Chem. Technol. Biotechnol. 81: 1669-1677). The pretreatment is performed preferably at 1-40% dry matter, e.g., 2-30% dry matter or 5-20% dry matter, and often the initial pH is increased by the addition of alkali such as sodium carbonate.

A modification of the wet oxidation pretreatment method, known as wet explosion (combination of wet oxidation and steam explosion) can handle dry matter up to 30%. In wet explosion, the oxidizing agent is introduced during pretreatment after a certain residence time. The pretreatment is then ended by flashing to atmospheric pressure (WO 2006/032282).

Ammonia fiber expansion (AFEX) involves treating the cellulosic material with liquid or gaseous ammonia at moderate temperatures such as 90-150° C. and high pressure such as 17-20 bar for 5-10 minutes, where the dry matter content can be as high as 60% (Gollapalli et al., 2002, Appl. Biochem. Biotechnol. 98: 23-35; Chundawat et al., 2007, Biotechnol. Bioeng. 96: 219-231; Alizadeh et al., 2005, Appl. Biochem. Biotechnol. 121: 1133-1141; Teymouri et al., 2005, Bioresource Technology 96: 2014-2018). During AFEX pretreatment cellulose and hemicelluloses remain relatively intact. Lignin-carbohydrate complexes are cleaved.

Organosolv pretreatment delignifies the cellulosic material by extraction using aqueous ethanol (40-60% ethanol) at 160-200° C. for 30-60 minutes (Pan et al., 2005, Biotechnol. Bioeng. 90: 473-481; Pan et al., 2006, Biotechnol. Bioeng. 94: 851-861; Kurabi et al., 2005, Appl. Biochem. Biotechnol. 121: 219-230). Sulphuric acid is usually added as a catalyst. In organosolv pretreatment, the majority of hemicellulose and lignin is removed.

Other examples of suitable pretreatment methods are described by Schell et al., 2003, Appl. Biochem. Biotechnol. 105-108: 69-85, and Mosier et al., 2005, supra, and U.S. Published Application 2002/0164730.

In one embodiment, the chemical pretreatment is preferably carried out as a dilute acid treatment, and more preferably as a continuous dilute acid treatment. The acid is typically sulfuric acid, but other acids may also be used, such as acetic acid, citric acid, nitric acid, phosphoric acid, tartaric acid, succinic acid, hydrogen chloride, or mixtures thereof. Mild acid treatment is conducted in the pH range of preferably 1-5, e.g., 1-4 or 1-2.5. In one embodiment, the acid concentration is in the range from preferably 0.01 to 10 wt. % acid, e.g., 0.05 to 5 wt. % acid or 0.1 to 2 wt. % acid. The acid is contacted with the cellulosic material and held at a temperature in the range of preferably 140-200° C., e.g., 165-190° C., for periods ranging from 1 to 60 minutes.

In another embodiment, pretreatment takes place in an aqueous slurry. In preferred embodiments, the cellulosic material is present during pretreatment in amounts preferably between 10-80 wt. %, e.g., 20-70 wt. % or 30-60 wt. %, such as around 40 wt. %. The pretreated cellulosic material may be unwashed or washed using any method known in the art, e.g., washed with water.

Mechanical Pretreatment or Physical Pretreatment: The term “mechanical pretreatment” or “physical pretreatment” refers to any pretreatment that promotes size reduction of particles. For example, such pretreatment may involve various types of grinding or milling (e.g., dry milling, wet milling, or vibratory ball milling).

The cellulosic material may be pretreated both physically (mechanically) and chemically. Mechanical or physical pretreatment may be coupled with steaming/steam explosion, hydrothermolysis, dilute or mild acid treatment, high temperature, high pressure treatment, irradiation (e.g., microwave irradiation), or combinations thereof. In one embodiment, high pressure means pressure in the range of preferably about 100 to about 400 psi, e.g., about 150 to about 250 psi. In another embodiment, high temperature means temperature in the range of about 100 to about 300° C., e.g., about 140 to about 200° C. In a preferred embodiment, mechanical or physical pretreatment is performed in a batch-process using a steam gun hydrolyzer system that uses high pressure and high temperature as defined above, e.g., a Sunds Hydrolyzer available from Sunds Defibrator AB, Sweden. The physical and chemical pretreatments may be carried out sequentially or simultaneously, as desired.

Accordingly, in a preferred embodiment, the cellulosic material is subjected to physical (mechanical) or chemical pretreatment, or any combination thereof, to promote the separation and/or release of cellulose, hemicellulose, and/or lignin.

Biological Pretreatment: The term “biological pretreatment” refers to any biological pretreatment that promotes the separation and/or release of cellulose, hemicellulose, and/or lignin from the cellulosic material. Biological pretreatment techniques may involve applying lignin-solubilizing microorganisms and/or enzymes (see, for example, Hsu, T.-A., 1996, Pretreatment of biomass, in Handbook on Bioethanol: Production and Utilization, Wyman, C. E., ed., Taylor & Francis, Washington, D.C., 179-212; Ghosh and Singh, 1993, Adv. Appl. Microbiol. 39: 295-333; McMillan, J. D., 1994, Pretreating lignocellulosic biomass: a review, in Enzymatic Conversion of Biomass for Fuels Production, Himmel, M. E., Baker, J. O., and Overend, R. P., eds., ACS Symposium Series 566, American Chemical Society, Washington, D.C., chapter 15; Gong, C. S., Cao, N. J., Du, J., and Tsao, G. T., 1999, Ethanol production from renewable resources, in Advances in Biochemical Engineering/Biotechnology, Scheper, T., ed., Springer-Verlag Berlin Heidelberg, Germany, 65: 207-241; Olsson and Hahn-Hagerdal, 1996, Enz. Microb. Tech. 18: 312-331; and Vallander and Eriksson, 1990, Adv. Biochem. Eng./Biotechnol. 42: 63-95).

### Hydrolysis (Saccharification)

In the hydrolysis step (i.e., saccharification step) the cellulosic material, e.g., pretreated lignocellulose, is hydrolyzed to break down cellulose and/or hemicellulose to fermentable sugars, such as glucose, cellobiose, xylose, xylulose, arabinose, mannose, galactose, and/or soluble oligosaccharides. In an embodiment, the hydrolysis is performed enzymatically using an enzyme composition comprising a cellulolytic composition and a hemicellulolytic composition. The hydrolysis is performed enzymatically by one or more enzyme compositions in one or more stages. As used herein “multi-stage saccharification” or “multi-stage hydrolysis” refers to a hydrolysis performed in two or more stages.

Processing of cellulosic material according to the present invention can be implemented using any conventional biomass processing apparatus configured to operate in accordance with embodiments of the invention.

A conventional apparatus can include a fed-batch stirred reactor, a batch stirred reactor, a continuous flow stirred reactor with ultrafiltration, and/or a continuous plug-flow column reactor (de Castilhos Corazza et al., 2003, Acta Scientiarum. Technology 25: 33-38; Gusakov and Sinitsyn, 1985, Enz. Microb. Technol. 7: 346-352), an attrition reactor (Ryu and Lee, 1983, Biotechnol. Bioeng. 25: 53-65). Additional reactor types include fluidized bed, upflow blanket, immobilized, and extruder type reactors for hydrolysis and/or fermentation.

The hydrolysis can be carried out as a continuous process, or series of batch and/or series of continuous processes, where the cellulosic or hemicellulosic material is fed gradually to, for example, an enzyme-containing hydrolysis solution. In an embodiment of the invention comprising a multi-stage hydrolysis, at least two stages are carried out in a single reactor. The hydrolysis may also be carried out as a series of batch and continuous processes.

Operation of multiple reactors in series allows for closer control of elements within each reactor, e.g., temperature, pH, mixing, concentration, and the like. Therefore in an embodiment of the invention comprising a multi-stage saccharification, at least two stages are carried out in separate reactors. In a preferred embodiment, each stage of a multi-stage saccharification is carried out in a separate reactor. In a further preferred embodiment, at least one stage in a multi-stage saccharification is carried out in a continuously operating reactor (e.g., CSTR). In a still further preferred embodiment, a continuous reactor in a multi-stage saccharification process is a continuously stirred tank reactor (CSTR) reactor, in series with at least one additional reactor. In another preferred embodiment, a continuously stirred reactor is followed in series with at least one additional reactor. In yet another preferred embodiment, a continuously stirred reactor is followed in series by at least one batch reactor.

Continuous operation, such as in use of a CSTR, provides advantages of continuous production and a steady state of operation once the reactor is running. Use of a continuous reactor permits management of high viscosity unhydrolyzed substrate, which also permits operation with higher total solids than might be available in a batch reactor. Semi-batch and semi-continuous operation may permit better control of environmental conditions and provide additional flexibility, compared to pure batch processes for selection of optimal conditions. For large scale hydrolysis processes, continuous operation is often preferred to eliminate downtime and to maximize production, though semi-batch and semi-continuous operation may also be used. However, as provided in Example 1, a gap in performance may be seen via a reduced sugar yield when a CSTR is used versus using a pure batch reactor.

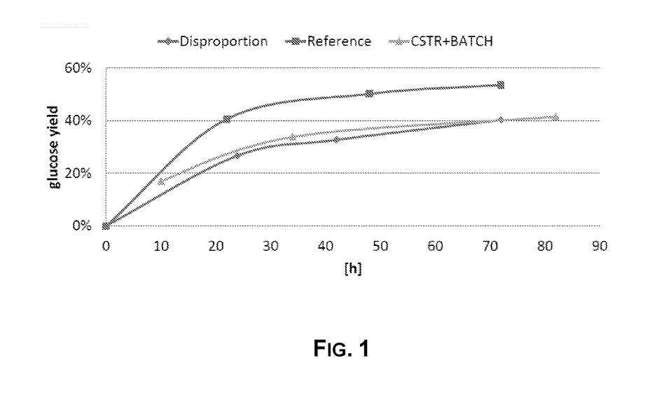

Example 1 provides a two stage hydrolysis of steam exploded wheat straw where a first stage is conducted in CSTR and a second stage is conducted in batch reactor, as compared to the hydrolysis of steam exploded wheat straw in a pure batch process. It is shown in FIG. 1 that a gap in yield is observed, where the process with a first stage CSTR presents a lower glucose yield than the yield from a pure batch process.

It was further evaluated by rotisserie study, as set forth in Example 2, whether addition of a hemicellulolytic composition in a batch process could serve to increase sugar yields. While previous studies have shown that xylanase supplementation of commercial cellulase compositions in batch processes can increase yield, such increases were obtained by substantially increasing the total amount of protein, thereby achieving a higher yield (Alvira, P., et al., Biotechnol. Prog., 2011, vol. 27, no. 4., p. 944-950; Qing, Q., et al., Biotech. for Biofuels, 2011, 4:18, p. 1-12.)

Example 2 provides a rotisserie scale study of the effects of adding a hemicellulolytic composition of xylanase and beta-xylosidase to pretreated arundo and wheat straw, by increasing the ratio of hemicellulolytic composition to a cellulolytic composition to maintain a steady level of total protein. The total protein loading was 7 mg of total enzyme protein per gram of cellulose in the cellulosic material in all tested combinations. It is seen that for arundo, the lowest percentage of hemicellulolytic composition resulted in the highest yield of both glucose and xylose.

For wheat straw, the increased percentage of hemicellulolytic composition resulted in the highest yield of xylose, but the glucose yield remained similar across all three tested fractions. From this rotisserie study, it was determined that increasing the percentage of hemicellulolytic composition does not provide a benefit to glucose yield obtained by batch hydrolysis of steam exploded wheat straw.

However, it was further examined whether increasing the ratio of hemicellulolytic composition to a cellulolytic composition while maintaining a steady level of total protein might differently affect a continuous process. Example 3 demonstrates that that with increased percentages of xylanase in an enzyme composition added to a first stage CSTR without significant elevation of the total protein levels, the gap seen in Example 1 can be minimized, if not eliminated. In Example 3, the 80:20 composition contained an amount of xylanase of about 102.0 U to about 213.5 U per gram of cellulosic material, particularly about 125.8 U per gram of cellulosic material and an amount of beta-xylosidase of about 1.2 U to about 3.99 U, particularly about 1.68 U per gram of cellulosic material.

As described herein, hydrolysis of cellulosic material is performed enzymatically by one or more enzyme compositions in one or more stages of hydrolysis. In an embodiment the invention provides processes including multi-stage saccharification including use of an enzyme composition comprising a hemicellulolytic composition in a ratio to a cellulolytic composition sufficient to increase sugar yields from a process including a continuous reactor, as compared to yields obtained from a process including the same reactor without use of such an enzyme composition. In a particular embodiment the increase in sugar yield from a process including a continuous reactor is sufficient to decrease or eliminate an observed gap in yield between a process including a continuous reactor and a pure batch process, where, without the addition of the composition comprising a hemicellulolytic composition the process including the continuous reactor has a lower sugar yield than the pure batch process.

As used herein, a hydrolysis process performed “without use of such an enzyme composition” may refer to a process performed without addition of a hemicellulolytic composition or may refer to a process performed with addition of an enzyme composition including both a cellulolytic composition and hemicellulolytic composition, but with ratios outside the range described herein, e.g., a ratio of cellulolytic composition to hemicellulolytic composition of from about 100:0 to more than about 85:15, or a ratio of cellulolytic composition to hemicellulolytic composition of from less than about 65:35 to about 0:100. Such may also refer to an enzyme composition including both a cellulolytic composition and hemicellulolytic composition, but with activity outside the range described herein, e.g., xylanase activity of less than about 12.75 U per g of cellulosic material or greater than about 457.5 U per g of cellulosic material, beta-xylosidase activity of less than about 0.15 U per g of cellulosic material or greater than about 8.55 U per g of cellulosic material or cellulase activity of less than about 3.15 U per g of cellulosic material or greater than about 99.0 U per g of cellulosic material.

In various embodiments, the enzyme composition may be added either before saccharification or after saccharification has been initiated.

Enzymatic hydrolysis (i.e., saccharification) is preferably carried out in a suitable aqueous environment under conditions that may be readily determined by one skilled in the art. In one embodiment, hydrolysis is performed under conditions suitable for the activity of the enzyme composition, preferably optimal for the enzyme composition.

The hydrolysis is generally performed in stirred-tank reactors or fermentors under controlled pH, temperature, and mixing conditions. Suitable process time, temperature and pH conditions may readily be determined by one skilled in the art. For example, the hydrolysis may last up to 200 hours, but is typically performed for preferably about 12 to about 120 hours, e.g., about 16 to about 72 hours or about 24 to about 48 hours. The temperature is in the range of preferably about 25° C. to about 70° C., e.g., about 30° C. to about 65° C., about 40° C. to about 60° C., or about 50° C. to about 55° C. The pH is in the range of preferably about 3 to about 8, e.g., about 3.5 to about 7, about 4 to about 6, or about 4.5 to about 5.5. The dry solids content is in the range of preferably about 5 to about 50 wt. %, e.g., about 10 to about 40 wt. % or about 15 to about 30 wt. %.

The present invention therefore relates to processes of multi-stage saccharification of a lignocellulosic material, the process comprising the steps of saccharifying a lignocellulosic material in a continuously operating reactor (e.g., CSTR) with an enzyme composition comprising a cellulolytic composition and a hemicellulolytic composition in a mass ratio of from about 85:15 to about 65:35 (cellulolytic composition:hemicellulolytic composition); and continuing saccharification in an additional reactor in series with the first reactor without further addition of enzymes to form a hydrolyzate wherein the hydrolyzate has a glucose and/or xylose yield that is improved as compared to the glucose and/or xylose yield from a process with a similar reactor setup, but without such enzyme composition. In an embodiment, the hemicellulolytic composition comprises an effective amount of xylanase of about 12.75 U to about 457.5 U, preferably about 102.0 U to about 213.5 U per g of the cellulosic material. In a further embodiment, the hemicellulolytic composition comprises an effective amount of beta-xylosidase of about 0.15 U to about 8.55 U, preferably about 1.2 U to about 3.99 U per g of the cellulosic material. In one embodiment, the processes further comprise recovering the hydrolyzate. Soluble products of degradation of the cellulosic material can be separated from insoluble cellulosic material using a method known in the art such as, for example, centrifugation, filtration, or gravity settling.

The present invention further relates to processes of improving a glucose or xylose yield of saccharification of a lignocellulosic material in a continuous process, the process comprising the steps of saccharifying a lignocellulosic material in a continuously operating reactor (e.g., CSTR) with an enzyme composition comprising a cellulolytic composition and a hemicellulolytic composition in a mass ratio of from about 85:15 to about 65:35 (cellulolytic composition:hemicellulolytic composition); and continuing saccharification in an additional reactor in series with the first reactor without further addition of enzymes to form a hydrolyzate wherein the hydrolyzate has a glucose yield or a xylose yield that are improved as compared to the yields from a process with a similar reactor setup, but without such enzyme composition. In an embodiment, the hemicellulolytic composition comprises an effective amount of xylanase of about 12.75 U to about 457.5 U, preferably about 102.0 U to about 213.5 U per g of the cellulosic material. In a further embodiment, the hemicellulolytic composition comprises an effective amount of beta-xylosidase of about 0.15 U to about 8.55 U, preferably about 1.2 U to about 3.99 U per g of the cellulosic material.

### Enzymes for Hydrolysis

The present invention relates to processes using an enzyme composition comprising a hemicellulolytic composition. Preferably the compositions are enriched in xylanase. As used herein the term “enriched” indicates that the xylanase activity of the composition has been increased by inclusion of more than 20% of a hemicellulolytic composition in the enzyme composition.

One or more (e.g., several) components of the enzyme composition may be native proteins, recombinant proteins, or a combination of native proteins and recombinant proteins. For example, one or more (e.g., several) components may be native proteins of a cell, which is used as a host cell to express recombinantly one or more (e.g., several) other components of the enzyme composition. It is understood herein that the recombinant proteins may be heterologous (e.g., foreign) and/or native to the host cell. One or more (e.g., several) components of the enzyme composition may be produced as monocomponents, which are then combined to form the enzyme composition. The enzyme composition may be a combination of multicomponent and monocomponent protein preparations. The compositions may be further combined with one or more additional enzyme compositions.

The enzymes used in processes of the present invention may be in any form suitable for use, such as, for example, a fermentation broth formulation or a cell composition, a cell lysate with or without cellular debris, a semi-purified or purified enzyme preparation, or a host cell as a source of the enzymes. The enzyme composition may be a dry powder or granulate, a non-dusting granulate, a liquid, a stabilized liquid, or a stabilized protected enzyme. Liquid enzyme preparations may, for instance, be stabilized by adding stabilizers such as a sugar, a sugar alcohol or another polyol, and/or lactic acid or another organic acid according to established processes.

The optimum amounts of the enzymes and polypeptides depend on several factors including, but not limited to, the mixture of cellulolytic enzymes and/or hemicellulolytic enzymes, the cellulosic material, the concentration of cellulosic material, the pretreatment of the cellulosic material, temperature, time, pH, and inclusion of a fermenting organism (e.g., for Simultaneous Saccharification and Fermentation).

In one embodiment the enzyme composition comprising cellulolytic composition and hemicellulolytic composition comprises an about 85:15 ratio of cellulolytic composition to a hemicellulolytic composition.

In another embodiment the enzyme composition comprising cellulolytic composition and hemicellulolytic composition comprises an about 80:20 ratio of cellulolytic composition to hemicellulolytic composition by mass. In another embodiment the enzyme composition comprising cellulolytic composition and hemicellulolytic composition comprises an about 75:25 ratio of cellulolytic composition to hemicellulolytic composition by mass. In another embodiment the enzyme composition comprising cellulolytic composition and hemicellulolytic composition comprises an about 70:30 ratio of cellulolytic composition to hemicellulolytic composition by mass. In another embodiment the enzyme composition comprising cellulolytic composition and hemicellulolytic composition comprises an about 65:35 ratio of cellulolytic composition to hemicellulolytic composition by mass. In another embodiment the enzyme composition comprising cellulolytic composition and hemicellulolytic composition comprises an about 60:40 ratio of cellulolytic composition to hemicellulolytic composition by mass. In another embodiment the enzyme composition comprising cellulolytic composition and hemicellulolytic composition comprises an about 55:45 ratio of cellulolytic composition to hemicellulolytic composition by mass. In another embodiment the enzyme composition comprising cellulolytic composition and hemicellulolytic composition comprises an about 50:50 ratio of cellulolytic composition to hemicellulolytic composition by mass. In another embodiment the enzyme composition comprising cellulolytic composition and hemicellulolytic composition comprises an about 45:55 ratio of cellulolytic composition to hemicellulolytic composition by mass. In another embodiment the enzyme composition comprising cellulolytic composition and hemicellulolytic composition comprises an about 40:60 ratio of cellulolytic composition to hemicellulolytic composition by mass. In a preferred embodiment, the combined cellulolytic composition and hemicellulolytic composition comprise 100% of the enzyme composition added in hydrolysis.

In one embodiment, an effective amount of enzyme composition to the cellulosic material is about about 0.5 to about 15 mg, e.g., about 0.5 to about 10 mg, about 0.5 to about 9 mg, about 0.5 to about 6 mg, or about 0.5 to about 5 mg per g of the cellulosic material per g of the cellulosic material. In a preferred embodiment the total enzyme of the enzyme composition comprising cellulolytic composition and hemicellulolytic composition is about 4 to about 10 mg, or about 4 to about 7 mg per g of the cellulosic material.

In a particular embodiment of the invention, though the enzyme composition comprising cellulolytic composition and hemicellulolytic composition may comprise varying amounts of each component, it is contemplated that the total protein of the resultant composition is about 4 to about 10 mg, or about 4 to about 7 mg per gram of the cellulosic material. Where either of the percentages of the cellulolytic composition or hemicellulolytic composition is increased relative to the whole of the enzyme composition, the other composition is adjusted such that the total protein of the resultant composition remains within the range of about about 4 to about 10 mg, or about 4 to about 7 mg per gram of the cellulosic material.

The enzyme composition may be present or added during hydrolysis (i.e., saccharification) in amounts effective from about 0.001 to about 5.0 wt % of solids (TS), more preferably from about 0.025 to about 4.0 wt % of solids, and most preferably from about 0.005 to about 2.0 wt % of solids (TS).

The enzymes in the enzyme composition may be derived or obtained from any suitable origin, including, archaeal, bacterial, fungal, yeast, plant, or animal origin. The term “obtained” also means herein that the enzyme may have been produced recombinantly in a host organism employing methods described herein, wherein the recombinantly produced enzyme is either native or foreign to the host organism or has a modified amino acid sequence, e.g., having one or more (e.g., several) amino acids that are deleted, inserted and/or substituted, i.e., a recombinantly produced enzyme that is a mutant and/or a fragment of a native amino acid sequence or an enzyme produced by nucleic acid shuffling processes known in the art.

Encompassed within the meaning of a native enzyme are natural variants and within the meaning of a foreign enzyme are variants obtained by, e.g., site-directed mutagenesis or shuffling.

Each polypeptide may be a bacterial polypeptide. For example, each polypeptide may be a Gram-positive bacterial polypeptide having enzyme activity, or a Gram-negative bacterial polypeptide having enzyme activity.

Each polypeptide may also be a fungal polypeptide, e.g., a yeast polypeptide or a filamentous fungal polypeptide.

Chemically modified or protein engineered mutants of polypeptides may also be used.

One or more (e.g., several) components of the enzyme composition may be a recombinant component, i.e., produced by cloning of a DNA sequence encoding the single component and subsequent cell transformed with the DNA sequence and expressed in a host (see, for example, WO 91/17243 and WO 91/17244). The host may be a heterologous host (enzyme is foreign to host), but the host may under certain conditions also be a homologous host (enzyme is native to host). Monocomponent cellulolytic proteins may also be prepared by purifying such a protein from a fermentation broth.

In a particular embodiment the enzyme composition comprises a cellulolytic composition comprising one or more (e.g., several) enzymes selected from the group consisting of an endoglucanase, a cellobiohydrolase, a beta glucosidase and an AA9 polypeptide having cellulolytic enhancing activity. In a further embodiment the cellulolytic composition comprises one or more hemicellulases.

Examples of bacterial endoglucanases that may be used in the present invention, include, but are not limited to, Acidothermus cellulolyticus endoglucanase (WO 91/05039; WO 93/15186; U.S. Pat. No. 5,275,944; WO 96/02551; U.S. Pat. No. 5,536,655; WO 00/70031; WO 05/093050), Erwinia carotovara endoglucanase (Saarilahti et al., 1990, Gene 90: 9-14), Thermobifida fusca endoglucanase III (WO 05/093050), and Thermobifida fusca endoglucanase V (WO 05/093050).

Examples of fungal endoglucanases that may be used in the present invention, include, but are not limited to, Aspergillus aculeatus endoglucanase (Ooi et al., 1990, Nucleic Acids Research 18: 5884), Aspergillus kawachii endoglucanase (Sakamoto et al., 1995, Current Genetics 27: 435-439), Fusarium oxysporum endoglucanase (GenBank:L29381), Humicola grisea var. thermoidea endoglucanase (GenBank:AB003107), Humicola insolens endoglucanase V, Melanocarpus albomyces endoglucanase (GenBank:MAL515703), Myceliophthora thermophila CBS 117.65 endoglucanase, Neurospora crassa endoglucanase (GenBank:XM_324477), Thermoascus aurantiacus endoglucanase I (Gen Bank:AF487830), Trichoderma reesei endoglucanase I (Penttila et al., 1986, Gene 45: 253-263), Trichoderma reesei Cel7B endoglucanase I (GenBank:M15665), Trichoderma reesei endoglucanase II (Saloheimo et al., 1988, Gene 63:11-22), Trichoderma reesei Cel5A endoglucanase II (GenBank:M19373), Trichoderma reesei endoglucanase III (Okada et al., 1988, Appl. Environ. Microbiol. 64: 555-563, GenBank:AB003694), Trichoderma reesei endoglucanase V (Saloheimo et al., 1994, Molecular Microbiology 13: 219-228, GenBank:Z33381), and Trichoderma reesei strain No. VTT-D-80133 endoglucanase (GenBank:M15665).

Examples of cellobiohydrolases useful in the present invention include, but are not limited to, Aspergillus aculeatus cellobiohydrolase II (WO 2011/059740), Aspergillus fumigatus cellobiohydrolase I (GENSEQP™ Accession No. AZI04842), Aspergillus fumigatus cellobiohydrolase II (GENSEQP™ Accession No. AZI04854), Chaetomium thermophilum cellobiohydrolase I, Chaetomium thermophilum cellobiohydrolase II, Humicola insolens cellobiohydrolase I, Myceliophthora thermophila cellobiohydrolase II (WO 2009/042871), Penicillium occitanis cellobiohydrolase I (GenBank:AY690482), Talaromyces emersonii cellobiohydrolase I (GenBank:AF439936), Thielavia hyrcanie cellobiohydrolase II (WO 2010/141325), Thielavia terrestris cellobiohydrolase II (CEL6A, WO 2006/074435), Trichoderma reesei cellobiohydrolase I, Trichoderma reesei cellobiohydrolase II, and Trichophaea saccata cellobiohydrolase II (WO 2010/057086).

Examples of beta-glucosidases useful in the present invention include, but are not limited to, beta-glucosidases from Aspergillus aculeatus (Kawaguchi et al., 1996, Gene 173: 287-288), Aspergillus fumigatus (WO 2005/047499), an Aspergillus fumigatus variant such as GENSEQP™ Accession No. AZU67153, Aspergillus niger (Dan et al., 2000, J. Biol. Chem. 275: 4973-4980), Aspergillus oryzae (WO 02/095014) or the fusion protein having beta-glucosidase activity disclosed in WO 2008/057637, Penicillium brasilianum IBT 20888 (WO 2007/019442 and WO 2010/088387), Thielavia terrestris (WO 2011/035029), Trichophaea saccata (WO 2007/019442) and Trichoderma reesei.

Other useful endoglucanases, cellobiohydrolases, and beta-glucosidases are disclosed in numerous Glycosyl Hydrolase families using the classification according to Henrissat, 1991, Biochem. J. 280: 309-316, and Henrissat and Bairoch, 1996, Biochem. J. 316: 695-696.

The amount of cellulase in an enzyme composition of the present invention may be determined as described in Example 4 and measured in U/mg total enzyme. Therefore, in an embodiment, the effective amount of cellulase in an enzyme composition of the present invention is about 3.15 U to about 99.0 U, preferably about 25.2 U to about 46.2 U per g of the cellulosic material.

In the processes of the present invention, any AA9 polypeptide may be used as a component of the enzyme composition.

Examples of AA9 polypeptides useful in the processes of the present invention include, but are not limited to, AA9 polypeptides from Aspergillus aculeatus (WO 2012/125925), Aspergillus fumigatus (WO 2010/138754), Aurantiporus alborubescens (WO 2012/122477), Chaetomium thermophilum (WO 2012/101206), Humicola insolens (WO 2012/146171), Malbranchea cinnamomea (WO 2012/101206), Myceliophthora thermophila (WO 2009/085935, WO 2009/085859, WO 2009/085864, WO 2009/085868, WO 2009/033071, WO 2012/027374, and WO 2012/068236), Penicillium pinophilum (WO 2011/005867), Penicillium thomii (WO 2012/122477), Penicillium sp. (emersonii) (WO 2011/041397 and WO 2012/000892), Talaromyces emersonii (WO 2012/000892), Talaromyces leycettanus (WO 2012/101206), Talaromyces stipitatus (WO 2012/135659), Talaromyces thermophilus (WO 2012/129697 and WO 2012/130950), Thermoascus aurantiacus (WO 2005/074656 and WO 2010/065830), Thermoascus crustaceous (WO 2011/041504), Thermoascus sp. (WO 2011/039319), Thermomyces lanuginosus (WO 2012/113340, WO 2012/129699, WO 2012/130964, and WO 2012/129699), Thielavia terrestris (WO 2005/074647, WO 2008/148131, and WO 2011/035027), Trametes versicolor (WO 2012/092676 and WO 2012/093149), Trichoderma reesei (WO 2007/089290 and WO 2012/149344), and Trichophaea saccata (WO 2012/122477).

In one embodiment, the AA9 polypeptide is used in the presence of a soluble activating divalent metal cation according to WO 2008/151043, e.g., manganese or copper.

In another embodiment, the AA9 polypeptide is used in the presence of a dioxy compound, a bicylic compound, a heterocyclic compound, a nitrogen-containing compound, a quinone compound, a sulfur-containing compound, or a liquor obtained from a pretreated cellulosic material such as pretreated corn stover (WO 2012/021394, WO 2012/021395, WO 2012/021396, WO 2012/021399, WO 2012/021400, WO 2012/021401, WO 2012/021408, and WO 2012/021410).

In one embodiment, such a compound is added at a molar ratio of the compound to glucosyl units of cellulose of about 10⁻⁶ to about 10, e.g., about 10⁻⁶ to about 7.5, about 10⁻⁶ to about 5, about 10⁻⁶ to about 2.5, about 10⁻⁶ to about 1, about 10⁻⁵ to about 1, about 10⁻⁵ to about 10⁻¹, about 10⁻⁴ to about 10⁻¹, about 10⁻³ to about 10⁻¹, or about 10⁻³ to about 10⁻². In another embodiment, an effective amount of such a compound is about 0.1 μM to about 1 M, e.g., about 0.5 μM to about 0.75 M, about 0.75 μM to about 0.5 M, about 1 μM to about 0.25 M, about 1 μM to about 0.1 M, about 5 μM to about 50 mM, about 10 μM to about 25 mM, about 50 μM to about 25 mM, about 10 μM to about 10 mM, about 5 μM to about 5 mM, or about 0.1 mM to about 1 mM.

The term “liquor” means the solution phase, either aqueous, organic, or a combination thereof, arising from treatment of a lignocellulose and/or hemicellulose material in a slurry, or monosaccharides thereof, e.g., xylose, arabinose, mannose, etc., under conditions as described in WO 2012/021401, and the soluble contents thereof. A liquor for cellulolytic enhancement of an AA9 polypeptide may be produced by treating a lignocellulose or hemicellulose material (or feedstock) by applying heat and/or pressure, optionally in the presence of a catalyst, e.g., acid, optionally in the presence of an organic solvent, and optionally in combination with physical disruption of the material, and then separating the solution from the residual solids. Such conditions determine the degree of cellulolytic enhancement obtainable through the combination of liquor and an AA9 polypeptide during hydrolysis of a cellulosic substrate by a cellulolytic composition. The liquor may be separated from the treated material using a method standard in the art, such as filtration, sedimentation, or centrifugation.

In one embodiment, an effective amount of the liquor to cellulose is about 10⁻⁶ to about 10 g per g of cellulose, e.g., about 10⁻⁶ to about 7.5 g, about 10⁻⁶ to about 5 g, about 10⁻⁶ to about 2.5 g, about 10⁻⁶ to about 1 g, about 10⁻⁵ to about 1 g, about 10⁻⁵ to about 10⁻¹ g, about 10⁻⁴ to about 10⁻¹ g, about 10⁻³ to about 10⁻¹ g, or about 10⁻³ to about 10⁻² g per g of cellulose.

In a particular embodiment the cellulolytic composition is derived from Trichoderma reesei, further comprising AA9 (GH61) polypeptide having cellulolytic enhancing activity set forth as SEQ ID NO: 2 in WO 2011/041397, a beta-glucosidase (SEQ ID NO: 2 in WO 2005/047499) variant (F100D, S283G, N456E, F512Y) set forth in WO 2012/044915; a CBH I set forth as SEQ ID NO: 6 in WO 2011/057140 and a CBH II set forth as SEQ ID NO: 18 in WO 2011/057140.

In a further embodiment the cellulolytic composition further comprises one or more hemicellulases. In an embodiment, the hemicellulase is preferably one or more (e.g., several) enzymes selected from the group consisting of an acetylmannan esterase, an acetylxylan esterase, an arabinanase, an arabinofuranosidase, a coumaric acid esterase, a feruloyl esterase, a galactosidase, a glucuronidase, a glucuronoyl esterase, a mannanase, a mannosidase, a xylanase, and a xylosidase.

In an embodiment the cellulolytic composition is or comprises a commercial cellulolytic composition. Examples of commercial cellulolytic compositions suitable for use in the present invention include, for example, CELLIC® CTec (Novozymes A/S), CELLIC® CTec2 (Novozymes A/S), CELLIC® Ctec3 (Novozymes A/S), CELLUCLAST® (Novozymes A/S), CELLUZYME™ (Novozymes A/S), CEREFLO® (Novo Nordisk A/S), and ULTRAFLO® (Novozymes A/S), ACCELLERASE® (Danisco US Inc.), LAMINEX® (Danisco US Inc.), SPEZYME® CP (Danisco US Inc.), ROHAMENT® 7069 W (AB Enzymes), FIBREZYME® LDI (Dyadic International, Inc.), FIBREZYME® LBR (Dyadic International, Inc.), or VISCOSTAR™ 150L (Dyadic International, Inc.).

In an embodiment the invention comprises use of an enzyme composition in processes described herein, wherein the enzyme composition comprises a cellulolytic composition. In a further embodiment the amount of enzyme in the cellulolytic composition is about 80% to about 35% of the total protein of the enzyme composition, e.g., about 79%, about 78%, about 77%, about 76%, about 75%, about 60%, about 55%, about 50%, about 45%, about 40%, about 39%, about 38%, about 37%, about 36%, about 35% of the total protein of the enzyme composition.

In a particular embodiment the enzyme composition comprises a hemicellulolytic composition comprising a xylanase and a beta-xylosidase. In a preferred embodiment the hemicellulolytic composition has endo-acting and exo-acting activity in hydrolysis of xylans. In a further embodiment the hemicellulolytic composition additionally comprises one or more (e.g., several) enzymes selected from the group consisting of acetylxylan esterases, feruloyl esterases, arabinofuranosidases, alpha-glucuronidases, and oxidoreductases.

Examples of xylanases useful in the processes of the present invention include, but are not limited to, xylanases from Aspergillus aculeatus (GENSEQP™ Accession No. AAR63790; WO 94/21785), Aspergillus fumigatus (WO 2006/078256), Penicillium pinophilum (WO 2011/041405), Penicillium sp. (WO 2010/126772), Talaromyces lanuginosus GH11 (WO 2012/130965), Talaromyces thermophilus GH11 (WO 2012/130950), Thielavia terrestris NRRL 8126 (WO 2009/079210), and Trichophaea saccata GH10 (WO 2011/057083).

Examples of beta-xylosidases useful in the processes of the present invention include, but are not limited to, beta-xylosidases from Aspergillus fumigatus (GENSEQP™ Accession No. AZI05042; WO 2013/028928), Neurospora crassa (SwissProt:Q7SOW4), Talaromyces emersonii (SwissProt:Q8X212), Trichoderma reesei (UniProtKB/TrEMBL:Q92458), and Trichoderma reesei such as the mature polypeptide of the mature polypeptide of GENSEQP™ Accession No. AZI04896.

Examples of acetylxylan esterases useful in the processes of the present invention include, but are not limited to, acetylxylan esterases from Aspergillus aculeatus (WO 2010/108918), Chaetomium globosum (UniProt:Q2GWX4), Chaetomium gracile (GeneSeqP:AAB82124), Humicola insolens DSM 1800 (WO 2009/073709), Hypocrea jecorina (WO 2005/001036), Myceliophtera thermophila (WO 2010/014880), Neurospora crassa (UniProt:q7s259), Phaeosphaeria nodorum (UniProt:Q0UHJ1), and Thielavia terrestris NRRL 8126 (WO 2009/042846).

Examples of feruloyl esterases (ferulic acid esterases) useful in the processes of the present invention include, but are not limited to, feruloyl esterases form Humicola insolens DSM 1800 (WO 2009/076122), Neosartorya fischeri (UniProt:A1D9T4), Neurospora crassa (UniProt:Q9HGR3), Penicillium aurantiogriseum (WO 2009/127729), and Thielavia terrestris (WO 2010/053838 and WO 2010/065448).

Examples of arabinofuranosidases useful in the processes of the present invention include, but are not limited to, arabinofuranosidases from Aspergillus niger (GeneSeqP:AAR94170), Humicola insolens DSM 1800 (WO 2006/114094 and WO 2009/073383), and M. giganteus (WO 2006/114094).

Examples of alpha-glucuronidases useful in the processes of the present invention include, but are not limited to, alpha-glucuronidases from Aspergillus clavatus (UniProt:alcc12), Aspergillus fumigatus (SwissProt:Q4WW45), Aspergillus niger (UniProt:Q96WX9), Aspergillus terreus (SwissProt:Q0CJP9), Humicola insolens (WO 2010/014706), Penicillium aurantiogriseum (WO 2009/068565), Talaromyces emersonii (UniProt:Q8X211), and Trichoderma reesei (UniProt:Q99024).

Examples of oxidoreductases useful in the processes of the present invention include, but are not limited to, Aspergillus lentilus catalase, Aspergillus fumigatus catalase, Aspergillus niger catalase, Aspergillus oryzae catalase, Humicola insolens catalase, Neurospora crassa catalase, Penicillium emersonii catalase, Scytalidium thermophilum catalase, Talaromyces stipitatus catalase, Thermoascus aurantiacus catalase, Coprinus cinereus laccase, Myceliophthora thermophila laccase, Polyporus pinsitus laccase, Pycnoporus cinnabarinus laccase, Rhizoctonia solani laccase, Streptomyces coelicolor laccase, Coprinus cinereus peroxidase, Soy peroxidase, Royal palm peroxidase.

The amount of xylanase may be determined as described in Example 5 and measured in U/mg total enzyme. Therefore in an embodiment, the effective amount of xylanase in an enzyme composition of the present invention is about 12.75 U to about 457.5 U, preferably about 102.0 U to about 213.5 U per g of the cellulosic material.

The amount of beta-xylosidase may be determined as described in Example 6 and measured in U/mg total enzyme. Therefore in an embodiment, the effective amount of beta-xylosidase in an enzyme composition of the present invention is about 0.15 U to about 8.55 U, preferably about 1.2 U to about 3.99 U per g of the cellulosic material.

In a particular embodiment the hemicellulolytic composition is derived from Trichoderma reesei, further comprising a xylanase (WO 2006/078256) and beta-xylosidase (WO 2011/057140).

In an embodiment the hemicellulolytic composition is or comprises a commercial hemicellulolytic composition. Examples of commercial hemicellulolytic compositions suitable for use in the present invention include, for example, SHEARZYME™ (Novozymes A/S), CELLIC® HTec (Novozymes A/S), CELLIC® HTec2 (Novozymes A/S), CELLIC® HTec3 (Novozymes A/S), VISCOZYME® (Novozymes A/S), ULTRAFLO® (Novozymes A/S), PULPZYME® HC (Novozymes A/S), MULTIFECT® Xylanase (Danisco US Inc.), ACCELLERASE® XY (Danisco US Inc.), ACCELLERASE® XC (Danisco US Inc.), ECOPULP® TX-200A (Roal Oy LLC), HSP 6000 Xylanase (DSM), DEPOL™ 333P (Biocatalysts Limit, Wales, UK), DEPOL™ 740L. (Biocatalysts Limit, Wales, UK), and DEPOL™ 762P (Biocatalysts Limit, Wales, UK), ALTERNA FUEL 100P (Dyadic), and ALTERNA FUEL 200P (Dyadic).

In an embodiment the invention comprises use of an enzyme composition in processes described herein, wherein the enzyme composition comprises a hemicellulolytic composition. In a further embodiment the amount of enzyme in the hemicellulolytic composition is about 20% to about 65% of the total protein of the enzyme composition, e.g., about 20%, about 21%, about 22%, about 23%, about 24%, about 25%, about 30%, about 35%, about 40%, about 45%, about 50%, about 55%, about 60%, about 61%, about 62%, about 63%, about 64%, about 65% of the total protein of the enzyme composition.

According to processes of the invention, an enzyme composition comprising a cellulolytic composition and a hemicellulolytic composition are present or added during hydrolysis (i.e., saccharification) of a lignocellulosic biomass. The enzyme composition may be added prior to hydrolysis or may be added during hydrolysis. In an embodiment, addition of the enzyme composition is sufficient to begin hydrolysis.

In one embodiment the cellulolytic composition and hemicellulolytic composition may be mixed or blended to form the enzyme composition prior to addition to the reactor. In another embodiment the cellulolytic composition and hemicellulolytic composition are added to a reactor simultaneously. In still another embodiment the cellulolytic composition and hemicellulolytic composition are added to a reactor sequentially. In a further embodiment, the enzyme composition, or component parts thereof, is added to a reactor before, concurrent with, or after addition of the lignocellulosic material to the reactor.

One or more (e.g., several) of the enzymes in the enzyme composition may be wild-type proteins expressed by the host strain, recombinant proteins, or a combination of wild-type proteins expressed by the host strain and recombinant proteins. For example, one or more (e.g., several) enzymes may be native proteins of a cell, which is used as a host cell to express recombinantly the enzyme composition.

The enzyme compositions may be prepared in accordance with methods known in the art and may be in the form of a liquid or a dry composition. The compositions may be stabilized in accordance with methods known in the art.

The enzyme compositions may result from a single fermentation or may be a blend of two or more fermentations, e.g., three, four, five, six, seven, etc. fermentations.

The enzyme compositions may be in any form suitable for use, such as, for example, a crude fermentation broth with or without cells removed, a cell lysate with or without cellular debris, a semi-purified or purified enzyme preparation, or a Trichoderma host cell as a source of the enzymes. The enzyme composition may be a dry powder or granulate, a non-dusting granulate, a liquid, a stabilized liquid, or a stabilized protected enzyme. Liquid enzyme preparations may, for instance, be stabilized by adding stabilizers such as a sugar, a sugar alcohol or another polyol, and/or lactic acid or another organic acid according to established processes.

The enzyme compositions may also be a fermentation broth formulation or a cell composition. The fermentation broth product further comprises additional ingredients used in the fermentation process, such as, for example, cells (including, the host cells containing the gene encoding the polypeptide of the present invention which are used to produce the polypeptide), cell debris, biomass, fermentation media and/or fermentation products. In some embodiments, the composition is a cell-killed whole broth containing organic acid(s), killed cells and/or cell debris, and culture medium.

The term “fermentation broth” refers to a preparation produced by cellular fermentation that undergoes no or minimal recovery and/or purification. For example, fermentation broths are produced when microbial cultures are grown to saturation, incubated under carbon-limiting conditions to allow protein synthesis (e.g., expression of enzymes by host cells) and secretion into cell culture medium. The fermentation broth can contain unfractionated or fractionated contents of the fermentation materials derived at the end of the fermentation. Typically, the fermentation broth is unfractionated and comprises the spent culture medium and cell debris present after the microbial cells (e.g., filamentous fungal cells) are removed, e.g., by centrifugation. In some embodiments, the fermentation broth contains spent cell culture medium, extracellular enzymes, and viable and/or nonviable microbial cells.

In an embodiment, the fermentation broth formulation and cell compositions comprise a first organic acid component comprising at least one 1-5 carbon organic acid and/or a salt thereof and a second organic acid component comprising at least one 6 or more carbon organic acid and/or a salt thereof. In a specific embodiment, the first organic acid component is acetic acid, formic acid, propionic acid, a salt thereof, or a mixture of two or more of the foregoing and the second organic acid component is benzoic acid, cyclohexanecarboxylic acid, 4-methylvaleric acid, phenylacetic acid, a salt thereof, or a mixture of two or more of the foregoing.

In one aspect, the composition contains an organic acid(s), and optionally further contains live cells, killed cells and/or cell debris. In one embodiment, the composition comprises live cells. In another embodiment, killed cells, and/or cell debris are removed from a cell-killed whole broth to provide a composition that is free of these components.

The fermentation broth formulations or cell compositions may further comprise a preservative and/or anti-microbial (e.g., bacteriostatic) agent, including, but not limited to, sorbitol, sodium chloride, potassium sorbate, and others known in the art.

The cell-killed whole broth or composition may contain the unfractionated contents of the fermentation materials derived at the end of the fermentation. Typically, the cell-killed whole broth or composition contains the spent culture medium and cell debris present after the microbial cells (e.g., filamentous fungal cells) are grown to saturation, incubated under carbon-limiting conditions to allow protein synthesis (e.g., expression of cellulase and/or glucosidase enzyme(s)). In some embodiments, the cell-killed whole broth or composition contains the spent cell culture medium, extracellular enzymes, and killed filamentous fungal cells. In some embodiments, the microbial cells present in the cell-killed whole broth or composition can be permeabilized and/or lysed using methods known in the art.

A whole broth or cell composition as described herein is typically a liquid slurry, but may contain insoluble components, such as killed cells, cell debris, culture media components, and/or insoluble enzyme(s). In some embodiments, insoluble components may be removed to provide a clarified liquid composition.

The whole broth formulations and cell compositions of the present invention may be produced by the methods described in WO 90/15861 or WO 2010/096673.

The fermentation may be any method of cultivation of a cell resulting in the expression or isolation of an enzyme or protein. Fermentation may, therefore, be understood as comprising shake flask cultivation, or small-scale or large-scale fermentation (including continuous, batch, fed-batch, or solid state fermentations) in laboratory or industrial fermentors performed in a suitable medium and under conditions allowing the enzyme to be expressed or isolated. The resulting enzymes produced by the methods described above may be recovered from the fermentation medium and purified by conventional procedures.

### Fermentation

The fermentable sugars obtained from the hydrolyzed cellulosic material may be fermented by one or more (e.g., several) fermenting microorganisms capable of fermenting the sugars directly or indirectly into a desired fermentation product.

“Fermentation” or “fermentation process” refers to any fermentation process or any process comprising a fermentation step. Fermentation processes also include fermentation processes used in the consumable alcohol industry (e.g., beer and wine), dairy industry (e.g., fermented dairy products), leather industry, and tobacco industry. The fermentation conditions depend on the desired fermentation product and fermenting organism and may easily be determined by one skilled in the art.

In the fermentation step, sugars, released from the cellulosic material as a result of the pretreatment and enzymatic hydrolysis steps, are fermented to a product, e.g., ethanol, by a fermenting organism, such as yeast. Hydrolysis (saccharification) and fermentation may be separate or simultaneous. Hydrolysis as described herein includes multi-stage hydrolysis. Where hydrolysis and fermentation are simultaneous, fermentation is carried out with one or more stages of hydrolysis.

Hydrolysis (saccharification) and fermentation, separate or simultaneous, include, but are not limited to, separate hydrolysis and fermentation (SHF); simultaneous saccharification and fermentation (SSF); simultaneous saccharification and co-fermentation (SSCF); hybrid hydrolysis and fermentation (HHF); separate hydrolysis and co-fermentation (SHCF); hybrid hydrolysis and co-fermentation (HHCF); and direct microbial conversion (DMC), also sometimes called consolidated bioprocessing (CBP). SHF uses separate process steps to first enzymatically hydrolyze the cellulosic material to fermentable sugars, e.g., glucose, cellobiose, and pentose monomers, and then ferment the fermentable sugars to ethanol. In SSF, the enzymatic hydrolysis of the cellulosic material and the fermentation of sugars to ethanol are combined in one step (Philippidis, G. P., 1996, Cellulose bioconversion technology, in Handbook on Bioethanol: Production and Utilization, Wyman, C. E., ed., Taylor & Francis, Washington, D.C., 179-212). SSCF involves the co-fermentation of multiple sugars (Sheehan and Himmel, 1999, Biotechnol. Prog. 15: 817-827). HHF involves a separate hydrolysis step, and in addition a simultaneous saccharification and hydrolysis step, which can be carried out in the same reactor. The steps in an HHF process can be carried out at different temperatures, i.e., high temperature enzymatic saccharification followed by SSF at a lower temperature that the fermentation strain can tolerate. DMC combines all three processes (enzyme production, hydrolysis, and fermentation) in one or more (e.g., several) steps where the same organism is used to produce the enzymes for conversion of the cellulosic material to fermentable sugars and to convert the fermentable sugars into a final product (Lynd et al., 2002, Microbiol. Mol. Biol. Reviews 66: 506-577). It is understood herein that any method known in the art comprising pretreatment, enzymatic hydrolysis (saccharification), fermentation, or a combination thereof, can be used in the practicing processes of the present invention.

Still further, the invention relates to processes of producing a fermentation product from a lignocellulosic material, the process comprising the steps of saccharifying the lignocellulosic material, comprising saccharifying a lignocellulosic material in a continuously operating reactor (e.g., CSTR) with an enzyme composition comprising a cellulolytic composition and a hemicellulolytic composition in a mass ratio of from about 85:15 to about 65:35; and continuing saccharification in an additional reactor in series with the first reactor without further addition of enzymes to form a hydrolyzate wherein the hydrolyzate has a glucose and/or xylose yield that is improved as compared to the glucose and/or xylose yield from a process with a similar reactor setup, but without such enzyme composition; and fermenting the hydrolyzate to produce a fermentation product.

The present invention also relates to processes of fermenting a lignocellulosic material, comprising: fermenting the cellulosic material with one or more (e.g., several) fermenting microorganisms, wherein the cellulosic material is saccharified in a continuously operating reactor (e.g., CSTR) with an enzyme composition comprising a cellulolytic composition and a hemicellulolytic composition in a mass ratio of from about 85:15 to about 65:35; and continuing saccharification in an additional reactor in series with the first reactor without further addition of enzymes to form a hydrolyzate. In an embodiment, the hemicellulolytic composition comprises an effective amount of xylanase of about 12.75 U to about 457.5 U, preferably about 102.0 U to about 213.5 U per g of the cellulosic material. In a further embodiment, the hemicellulolytic composition comprises an effective amount of beta-xylosidase of about 0.15 U to about 8.55 U, preferably about 1.2 U to about 3.99 U per g of the cellulosic material. In one embodiment, the fermenting of the cellulosic material produces a fermentation product. In another embodiment, the processes further comprise recovering the fermentation product from the fermentation.

Any suitable hydrolyzed cellulosic material may be used in the fermentation step in practicing the present invention. The material is generally selected based on economics, i.e., costs per equivalent sugar potential, and recalcitrance to enzymatic conversion.

The term “fermentation medium” is understood herein to refer to a medium before the fermenting microorganism(s) is (are) added, such as, a medium resulting from a saccharification process, as well as a medium used in a simultaneous saccharification and fermentation process (SSF).

Suitable fermenting organisms used according of processes of the invention are described below in the “Fermenting Organism”-section below

### Fermenting Organism

“Fermenting organism” or “fermenting microorganism” refers to any microorganism, including bacterial and fungal organisms, suitable for use in a desired fermentation process to produce a fermentation product. The fermenting organism may be hexose (i.e., C₆) and/or pentose (C₅) fermenting organisms, or a combination thereof. Both hexose and pentose fermenting organisms are well known in the art. Suitable fermenting organisms are able to ferment, i.e., convert, sugars, such as glucose, xylose, xylulose, arabinose, maltose, mannose, galactose, and/or oligosaccharides, directly or indirectly into the desired fermentation product. Examples of bacterial and fungal fermenting organisms producing ethanol are described by Lin et al., 2006, Appl. Microbiol. Biotechnol. 69: 627-642.

Examples of fermenting microorganisms that can ferment C₆ sugars include bacterial and fungal organisms, such as yeast. Yeast include strains of Candida, Kluyveromyces, and Saccharomyces, e.g., Candida sonorensis, Kluyveromyces marxianus, and Saccharomyces cerevisiae. Preferred yeast includes strains of the Saccharomyces spp., preferably Saccharomyces cerevisiae.

Examples of fermenting organisms that can ferment C₅ sugars include bacterial and fungal organisms, such as yeast. Preferred C₅ fermenting yeast include strains of Pichia, preferably Pichia stipitis, such as Pichia stipitis CBS 5773; strains of Candida, preferably Candida boidinii, Candida brassicae, Candida sheatae, Candida diddensii, Candida pseudotropicalis, or Candida utilis. Organisms not capable of fermenting pentose sugars, such as xylose and arabinose, may be genetically modified to do so by methods known in the art.

Examples of bacteria that can efficiently ferment hexose and pentose to ethanol include, for example, Bacillus coagulans, Clostridium acetobutylicum, Clostridium thermocellum, Clostridium phytofermentans, Geobacillus sp., Thermoanaerobacter saccharolyticum, and Zymomonas mobilis (Philippidis, 1996, supra).

Other fermenting organisms include strains of Bacillus, such as Bacillus coagulans; Candida, such as C. sonorensis, C. methanosorbosa, C. diddensiae, C. parapsilosis, C. naedodendra, C. blankii, C. entomophilia, C. brassicae, C. pseudotropicalis, C. boidinii, C. utilis, and C. scehatae; Clostridium, such as C. acetobutylicum, C. thermocellum, and C. phytofermentans; E. coli, especially E. coli strains that have been genetically modified to improve the yield of ethanol; Geobacillus sp.; Hansenula, such as Hansenula anomala; Klebsiella, such as K. oxytoca; Kluyveromyces, such as K. marxianus, K. lactis, K. thermotolerans, and K. fragilis; Schizosaccharomyces, such as S. pombe; Thermoanaerobacter, such as Thermoanaerobacter saccharolyticum; and Zymomonas, such as Zymomonas mobilis.

Commercially available yeast suitable for ethanol production include, e.g., BIO-FERM® AFT and XR, ETHANOL RED® yeast, FALI®, FERMIOL®, GERT STRAND™ (Gert Strand AB, Sweden), SUPERSTART™ and THERMOSACC® fresh yeast.

In an embodiment, the fermenting organism has been genetically modified to provide the ability to ferment pentose sugars, such as xylose utilizing, arabinose utilizing, and xylose and arabinose co-utilizing microorganisms.

The cloning of heterologous genes into various fermenting microorganisms has led to the construction of organisms capable of converting hexoses and pentoses to ethanol (cofermentation) (Chen and Ho, 1993, Appl. Biochem. Biotechnol. 39-40: 135-147; Ho et al., 1998, Appl. Environ. Microbiol. 64: 1852-1859; Kotter and Ciriacy, 1993, Appl. Microbiol. Biotechnol. 38: 776-783; Walfridsson et al., 1995, Appl. Environ. Microbiol. 61: 4184-4190; Kuyper et al., 2004, FEMS Yeast Research 4: 655-664; Beall et al., 1991, Biotech. Bioeng. 38: 296-303; Ingram et al., 1998, Biotechnol. Bioeng. 58: 204-214; Zhang et al., 1995, Science 267: 240-243; Deanda et al., 1996, Appl. Environ. Microbiol. 62: 4465-4470; WO 03/062430).

It is well known in the art that the organisms described above may also be used to produce other substances, as described herein.

The fermenting organism is typically added to the degraded cellulosic material or hydrolyzate and the fermentation is performed for about 8 to about 96 hours, such as about 24 to about 60 hours. The temperature is typically between about 26° C. to about 60° C., in particular about 32° C. or 50° C., and at about pH 3 to about pH 8, such as around pH 4-5, 6, or 7.

In one embodiment, the yeast and/or another microorganism are applied to the degraded cellulosic material and the fermentation is performed for about 12 to about 96 hours, such as typically 24-60 hours. In another embodiment, the temperature is preferably between about 20° C. to about 60° C., e.g., about 25° C. to about 50° C., about 32° C. to about 50° C., or about 32° C. to about 50° C., and the pH is generally from about pH 3 to about pH 7, e.g., about pH 4 to about pH 7. However, some fermenting organisms, e.g., bacteria, have higher fermentation temperature optima. Yeast or another microorganism is preferably applied in amounts of approximately 10⁵ to 10¹², preferably from approximately 10⁷ to 10¹⁰, especially approximately 2×10⁸ viable cell count per ml of fermentation broth. Further guidance in respect of using yeast for fermentation may be found in, e.g., “The Alcohol Textbook” (Editors K. Jacques, T. P. Lyons and D. R. Kelsall, Nottingham University Press, United Kingdom 1999), which is hereby incorporated by reference.

For ethanol production, following the fermentation the fermented slurry may be distilled to extract the ethanol. The ethanol obtained according to processes of the invention may be used as, e.g., fuel ethanol, drinking ethanol, i.e., potable neutral spirits, or industrial ethanol.

### Fermentation Stimulators

A fermentation stimulator may be used in combination with any of the processes described herein to further improve the fermentation process, and in particular, the performance of the fermenting microorganism, such as, rate enhancement and ethanol yield. A “fermentation stimulator” refers to stimulators for growth of the fermenting microorganisms, in particular, yeast. Preferred fermentation stimulators for growth include vitamins and minerals. Examples of vitamins include multivitamins, biotin, pantothenate, nicotinic acid, meso-inositol, thiamine, pyridoxine, para-aminobenzoic acid, folic acid, riboflavin, and Vitamins A, B, C, D, and E. See, for example, Alfenore et al., Improving ethanol production and viability of Saccharomyces cerevisiae by a vitamin feeding strategy during fed-batch process, Springer-Verlag (2002), which is hereby incorporated by reference. Examples of minerals include minerals and mineral salts that can supply nutrients comprising P, K, Mg, S, Ca, Fe, Zn, Mn, and Cu.

### Fermentation Products

Processes of the present invention can be used to saccharify the lignocellulosic material to fermentable sugars and to convert the fermentable sugars to many useful fermentation products, e.g., fuel (ethanol, n-butanol, isobutanol, biodiesel, jet fuel) and/or platform chemicals (e.g., acids, alcohols, ketones, gases, oils, and the like). The production of a desired fermentation product from the cellulosic material typically involves pretreatment, enzymatic hydrolysis (saccharification), and fermentation.

A fermentation product may be any substance derived from the fermentation. The fermentation product may be, without limitation, an alcohol (e.g., arabinitol, n-butanol, isobutanol, ethanol, glycerol, methanol, ethylene glycol, 1,3-propanediol [propylene glycol], butanediol, glycerin, sorbitol, and xylitol); an alkane (e.g., pentane, hexane, heptane, octane, nonane, decane, undecane, and dodecane), a cycloalkane (e.g., cyclopentane, cyclohexane, cycloheptane, and cyclooctane), an alkene (e.g., pentene, hexene, heptene, and octene); an amino acid (e.g., aspartic acid, glutamic acid, glycine, lysine, serine, and threonine); a gas (e.g., methane, hydrogen (H₂), carbon dioxide (CO₂), and carbon monoxide (CO)); isoprene; a ketone (e.g., acetone); an organic acid (e.g., acetic acid, acetonic acid, adipic acid, ascorbic acid, citric acid, 2,5-diketo-D-gluconic acid, formic acid, fumaric acid, glucaric acid, gluconic acid, glucuronic acid, glutaric acid, 3-hydroxypropionic acid, itaconic acid, lactic acid, malic acid, malonic acid, oxalic acid, oxaloacetic acid, propionic acid, succinic acid, and xylonic acid); and polyketide. The fermentation product may also be protein as a high value product.

In one embodiment, the fermentation product is an alcohol. The term “alcohol” encompasses a substance that contains one or more hydroxyl moieties. The alcohol may be, but is not limited to, n-butanol, isobutanol, ethanol, methanol, arabinitol, butanediol, ethylene glycol, glycerin, glycerol, 1,3-propanediol, sorbitol, xylitol. See, for example, Gong et al., 1999, Ethanol production from renewable resources, in Advances in Biochemical Engineering/Biotechnology, Scheper, T., ed., Springer-Verlag Berlin Heidelberg, Germany, 65: 207-241; Silveira and Jonas, 2002, Appl. Microbiol. Biotechnol. 59: 400-408; Nigam and Singh, 1995, Process Biochemistry 30(2): 117-124; Ezeji et al., 2003, World Journal of Microbiology and Biotechnology 19(6): 595-603.

In another embodiment, the fermentation product is an alkane. The alkane may be an unbranched or a branched alkane. The alkane may be, but is not limited to, pentane, hexane, heptane, octane, nonane, decane, undecane, or dodecane.

In another embodiment, the fermentation product is a cycloalkane. The cycloalkane may be, but is not limited to, cyclopentane, cyclohexane, cycloheptane, or cyclooctane.

In another embodiment, the fermentation product is an alkene. The alkene may be an unbranched or a branched alkene. The alkene may be, but is not limited to, pentene, hexene, heptene, or octene.

In another embodiment, the fermentation product is an amino acid. The organic acid may be, but is not limited to, aspartic acid, glutamic acid, glycine, lysine, serine, or threonine. See, for example, Richard and Margaritis, 2004, Biotechnology and Bioengineering 87(4): 501-515.

In another embodiment, the fermentation product is a gas. The gas may be, but is not limited to, methane, H₂, CO₂, or CO. See, for example, Kataoka et al., 1997, Water Science and Technology 36(6-7): 41-47; and Gunaseelan, 1997, Biomass and Bioenergy 13(1-2): 83-114.

In another embodiment, the fermentation product is isoprene.

In another embodiment, the fermentation product is a ketone. The term “ketone” encompasses a substance that contains one or more ketone moieties. The ketone may be, but is not limited to, acetone.

In another embodiment, the fermentation product is an organic acid. The organic acid may be, but is not limited to, acetic acid, acetonic acid, adipic acid, ascorbic acid, citric acid, 2,5-diketo-D-gluconic acid, formic acid, fumaric acid, glucaric acid, gluconic acid, glucuronic acid, glutaric acid, 3-hydroxypropionic acid, itaconic acid, lactic acid, malic acid, malonic acid, oxalic acid, propionic acid, succinic acid, or xylonic acid. See, for example, Chen and Lee, 1997, Appl. Biochem. Biotechnol. 63-65: 435-448.

In another embodiment, the fermentation product is polyketide.

### Recovery

The fermentation product(s) may be optionally recovered from the fermentation medium using any method known in the art including, but not limited to, chromatography, electrophoretic procedures, differential solubility, distillation, or extraction. For example, alcohol is separated from the fermented cellulosic material and purified by conventional methods of distillation. Ethanol with a purity of up to about 96 vol. % may be obtained, which may be used as, for example, fuel ethanol, drinking ethanol, i.e., potable neutral spirits, or industrial ethanol.

The present invention is further defined by the following paragraphs:

[1] A process of multi-stage saccharification of a lignocellulosic material, the process comprising the steps of:

a) saccharifying a lignocellulosic material in a continuously stirred tank reactor (CSTR) with an enzyme composition comprising a cellulolytic composition and a hemicellulolytic composition in a mass ratio of from about 85:15 to about 65:35 (cellulolytic composition:hemicellulolytic composition) and

b) continuing saccharification in an additional reactor in series with the CSTR without further addition of enzymes to form a hydrolyzate wherein the hydrolyzate has a glucose or xylose yield that is improved as compared to the glucose or xylose yield from a process without addition of such enzyme composition.

[2] A process of producing a fermentation product from a lignocellulosic material, the process comprising the steps of:

a) saccharifying the lignocellulosic material, comprising:
1) saccharifying a lignocellulosic material in a continuously stirred tank reactor (CSTR) with an enzyme composition comprising a cellulolytic composition and a hemicellulolytic composition in a mass ratio of from about 85:15 to about 65:35 (cellulolytic composition:hemicellulolytic composition); and
2) continuing saccharification in an additional reactor in series with the CSTR without further addition of enzymes to form a hydrolyzate wherein the hydrolyzate has a glucose or xylose yield that is improved as compared to the glucose or xylose yield from a process without addition of such enzyme composition; and
b) fermenting the hydrolyzate to produce a fermentation product.
   [3] A process of improving a glucose or xylose yield of saccharification of a lignocellulosic material in a continuously stirred tank reactor (CSTR), the process comprising the steps of:

a) saccharifying a lignocellulosic material in a CSTR with an enzyme composition comprising a cellulolytic composition and a hemicellulolytic composition in a mass ratio of from about 85:15 to about 65:35 (cellulolytic composition:hemicellulolytic composition); and

b) continuing saccharification in an additional reactor in series with the CSTR reactor without further addition of enzymes to form a hydrolyzate wherein the hydrolyzate has a glucose yield or a xylose yield that are improved as compared to the yields from a process without addition of such enzyme composition.

[4] The process of any of paragraphs 1 to 3, wherein the pretreated lignocellulosic material has been subjected to a pretreatment method selected from steam explosion and liquid hot water treatment, or a combination thereof.
[5] The process of any of paragraphs 1 to 3, wherein the lignocellulosic material has been subjected to a pretreatment selected from chemical pretreatment and mechanical pretreatment, but not subjected to steam explosion or liquid hot water treatment.
[6] The process of any of paragraphs 1 to 5, wherein the lignocellulosic material is wheat straw.
[7] The process of any of paragraphs 1 to 6, wherein the lignocellulosic material is steam exploded wheat straw.
[8] The process of any of paragraphs 1 to 7, wherein the enzyme composition comprises an about 85:15 ratio of cellulolytic composition to hemicellulolytic composition.
[9] The process of any of paragraphs 1 to 7, wherein the enzyme composition comprises an about 80:20 ratio of cellulolytic composition to hemicellulolytic composition.
[10] The process of any of paragraphs 1 to 7, wherein the enzyme composition comprises an about 70:30 ratio of cellulolytic composition to hemicellulolytic composition.
[11] The process of any of paragraphs 1 to 10, wherein the cellulolytic enzyme composition comprises an AA9, a beta-glucosidase, a CBH I and a CBH II.
[12] The process of any of paragraphs 1 to 11, wherein the hemicellulolytic enzyme composition comprises a xylanase and a beta-xylosidase.
[13] The process of any of paragraphs 1 to 12, wherein the saccharification is performed at a temperature of about 40° C. to about 60° C.
[14] The process of any of paragraphs 1 to 13, wherein the saccharification is performed at a temperature of about 50° C. to about 55° C.
[15] The process of any of paragraphs 1 to 14, wherein the saccharification is performed at a pH of about 4 to about 6.
[16] The process of any of paragraphs 1 to 15, wherein the saccharification is performed at a pH of about 4.5 to about 5.5.
[17] The process of any of paragraphs 1 to 16, wherein the additional reactor is a batch reactor.
[18] The process of any of paragraphs 1 to 17, wherein the additional reactor is a CSTR.
[19] The process of any of paragraphs 1 to 18, wherein the amount of xylanase in the enzyme composition is about 12.75 U to about 457.5 U per gram of the lignocellulosic material.
[20] The process of any of paragraphs 1 to 19, wherein the amount of beta-xylosidase in the enzyme composition is about 0.15 U to about 8.55 U per gram of the lignocellulosic material.
[21] The process of any of paragraphs 1 to 20, wherein the amount of cellulase in the enzyme composition is about 3.15 U to about 99.0 U per gram of the lignocellulosic material.

The present invention is further described by the following examples that should not be construed as limiting the scope of the invention.

The following are referred to in the examples:

Cellulolytic composition A (“CPrepA”): Cellulolytic composition derived from Trichoderma reesei further comprising AA9 (GH61) polypeptide having cellulolytic enhancing activity of SEQ ID NO: 10, a beta-glucosidase of SEQ ID NO: 4, a cellobiohydrolase I of SEQ ID NO: 6, a cellobiohydrolase II of SEQ ID NO: 8 herein, a xylanase of SEQ ID NO: 12 and a beta-xylosidase of SEQ ID NO: 14.

Cellulolytic composition B (“CPrepB”): Cellulolytic composition derived from Trichoderma reesei further comprising AA9 (GH61) polypeptide having cellulolytic enhancing activity of SEQ ID NO: 10, a beta-glucosidase of SEQ ID NO: 4, a cellobiohydrolase I of SEQ ID NO: 6 herein, and a cellobiohydrolase II of SEQ ID NO: 8.

Xylanase Enzyme Preparation (“XPrep”): Hemicellulolytic composition from Trichoderma reesei, further comprising a xylanase of SEQ ID NO: 12 and a beta-xylosidase of SEQ ID NO: 14.

The invention described and claimed herein is not to be limited in scope by the specific aspects or embodiments herein disclosed, since such are intended as illustrations of several aspects or embodiments of the invention. Any equivalent aspects or embodiments are intended to be within the scope of this invention. Indeed, various modifications of the invention in addition to those shown and described herein will become apparent to those skilled in the art from the foregoing description. Such modifications are also intended to fall within the scope of the appended claims. In the case of conflict, the present disclosure including definitions will control.

### EXAMPLES

### Example 1

### Comparison of Two Stage Hydrolysis with First Step of CSTR Vs. Batch

Wheat straw was introduced into a continuous reactor and subjected to a soaking treatment at a temperature of 158° C. for 65 minutes. The soaked mixture was separated in a soaked liquid and a fraction containing the solid soaked raw material by means of a press. The fraction containing the solid soaked raw material was subjected to steam explosion at a temperature of 200° C. for a time of 4 minutes to produce a solid stream.

Soaked liquid was subjected to a concentration step by means of a membrane filtration step, which also removes a portion of acetic acid. First, soaked liquids were subjected to a preliminary pre-separation step to remove solids, by means of centrifugation and macro filtration (bag filter with filter size of 1 micron). Centrifugation was performed by means of an Alfa Laval CLARA 80 centrifuge at 8000 rpm. The soaked liquid was then subjected to concentration by means of a Alfa Laval 2.5″ equipment (membrane code NF99 2517/48), operated at a VCR (Volume Concentration Ratio) of 2.5.

The pre-treatment, including concentration step, produced a soaked liquid and a solid stream in a ratio of liquid stream:solid stream by weight of 1:1. The soaked liquid and the solid stream were used as pre-treated wheat straw material in the following enzymatic hydrolysis experiments.

The dry matter of the soaked liquid after concentration was 10%. pH of the solid stream was 4 and pH of the liquid stream was 4.

The pretreated wheat straw was subjected to one of two hydrolysis reactions: two stage hydrolysis, with the first step in CSTR and the second step in batch, as compared to a pure batch hydrolysis.

A first stage CSTR contained a total reaction mass of 100 kg and was operated by discharging 10 kg of hydrolysis reaction material every hour and immediately adding 10 kg of pre-treated wheat straw material and water as 3.2 kg soaked liquid, 4 kg solid stream and 2.8 kg water. At the time of each addition of new material to the CSTR, 35 g of CPrepA was added to the CSTR at a dose of 5.7% (weight/weight glucan). The CSTR retention time was 10 hour. pH was controlled at a targeted 5.2 in the CSTR with additions of 2M sodium hydroxide. The subsequent batch hydrolysis was performed in a Labfors 5 BioEtOH reactor (Infors AG, Switzerland). The reaction in the CSTR was performed at 18% dry matter, 50° C. and pH 5.0 and the reaction in the Labfors 5 BioEtOH reactor was performed at 18% dry matter, 50° C. and pH 5.0. No additional enzymes were added in the subsequent stage batch hydrolysis.

The pure batch hydrolysis was performed in a mixed tank reactor filled with a total of 15 kg reaction mass. CPrepA was added at the start of the reaction in a dose of 5.6% (weight/weight glucan). The reaction was performed at 21% dry matter, 50° C. and pH 5.0.

The hydrolysis performance was evaluated in terms of glucose yield and xylose yield. The total glucose and xylose concentrations from each reaction was determined by HPLC. Glucose yield is the percent ratio of the amount of glucose in the hydrolyzed mixture to the amount of glucans in the pretreated streams, expressed as glucose equivalents. Glucose equivalents were calculated including insoluble glucans, gluco-oligomers, cellobiose and glucose, present in both the solid and liquid of the lignocellulosic biomass, taking into account the different molecular weights. Equivalently, xylose yield is the percent ratio of the amount of xylose in the hydrolyzed mixture to the amount of xylans in the pretreated streams, expressed as xylose equivalents. Xylose equivalents were calculated including insoluble xylans, xylooligomers, xylobiose and xylose, present in both the solid and liquid of the lignocellulosic biomass, taking into account the different molecular weights.

Table 1 shows glucose and xylose yield at different time points of the enzymatic hydrolysis of pre-treated wheat straw using two-stage enzyme addition in a continuously stirred tank reactor followed by hydrolysis in batch (CSTR+batch) compared to hydrolysis in only batch (pure batch) with CPrepA.

A lower glucose yield was obtained with a first stage hydrolysis in a CSTR, followed by second stage hydrolysis in a batch reactor, as compared to a pure batch hydrolysis. Results are set forth in Table 1 and illustrated in FIG. 1. The hydrolysis of the CSTR+batch reaction reached a glucose yield of 42% and a xylose yield of 62% after 82 h of reaction time compared to a glucose yield of 55% and a xylose yield of 65% for the pure batch hydrolysis after 72 h of reaction time. These yields illustrated that resulting sugar yields are lower in first stage CSTR as compared to pure batch.

TABLE 1

| | | Time (h) | | | |
|---|---|---|---|---|---|
| | | 10 h | 34 h | 58 h | 82 h |
| CSTR + batch | Glucose yield | 17% | 34% | 38% | 42% |
| | Xylose yield | 30% | 50% | 56% | 62% |
| | | Time (h) | | | |
| | | 0 h | 24 h | 48 h | 72 h |
| Pure batch | Glucose yield | 0% | 41% | 50% | 53% |
| | Xylose yield | 2% | 52% | 62% | 66% |

Analytical measurements were performed according to the following standards issued by the National Renewable Energy Laboratory (NREL):

Determination of Structural Carbohydrates and Lignin in Biomass; Laboratory Analytical Procedure (LAP) Issue Date: Apr. 25, 2008; Technical Report NREL/TP-510-42618 Revised April 2008

Determination of Extractives in Biomass; Laboratory Analytical Procedure (LAP) Issue Date: Jul. 17, 2005; Technical Report NREL/TP-510-42619 January 2008

Preparation of Samples for Compositional Analysis; Laboratory Analytical Procedure (LAP) Issue Date: Sep. 28, 2005; Technical Report NREL/TP-510-42620 January 2008

Determination of Total Solids in Biomass and Total Dissolved Solids in Liquid Process Samples; Laboratory Analytical Procedure (LAP) Issue Date: Mar. 31, 2008; Technical Report NREL/TP-510-42621 Revised March 2008

Determination of Ash in Biomass; Laboratory Analytical Procedure (LAP) Issue Date: Jul. 17, 2005; Technical Report NREL/TP-510-42622 January 2008

Determination of Sugars, By products, and Degradation Products in Liquid Fraction Process Samples; Laboratory Analytical Procedure (LAP) Issue Date: Dec. 8, 2006; Technical Report NREL/TP-510-42623 January 2008

Determination of Insoluble Solids in Pretreated Biomass Material; Laboratory Analytical Procedure (LAP) Issue Date: Mar. 21, 2008; NREL/TP-510-42627 March 2008

### Example 2

### Addition of Xylanase-Containing Enzyme Composition and Effect on Glucose Yield

Arundo donax (giant reed) and wheat straw were each pretreated in a two-stage steam explosion reactor. The wheat straw was pretreated by cooking in a two-stage process. In the first stage cook, the temperature was maintained at 158° C. for 65 minutes, and the liquid was squeezed from the material after the first stage cook. In the second stage cook, the squeezed material (dry solids) was subjected to a temperature of 195° C. for 4 minutes. The liquid that was squeezed out after the first cook and the solids from the second cook were combined to form pretreated wheat straw slurry

30 mM sodium acetate was added to the material in order to increase the pH buffering capacity and reduce pH drift during the trial. Total solids in the final blend of cellulosic material with enzymes was 15% for arundo and 12% for wheat straw.

The cellulosic material was mixed, pH adjusted, and equilibrated as follows. Liquor, water, and 1M sodium acetate buffer were mixed and adjusted to pH 5.6 by adding 50% sodium hydroxide. Solids were added and the material was carefully mixed and adjusted to pH 5.5. The material was incubated at 60° C. to equilibrate over night. pH was checked and re-adjusted the following day.

Cellulolytic Enzyme Composition B (“CPrepB”) was blended with Xylanase Enzyme Composition (“XPrep”) in ratios of 90:10, 80:20 and 70:30 and the blends were used for hydrolysis of the prepared cellulosic material. The applied dosage was 7 mg of total enzyme protein per gram of cellulose in the cellulosic material.

Enzymatic hydrolysis of the cellulosic material was carried out as follows. 19 grams of cellulosic material and 1 ml enzyme dilution were added to 50 mL polycarbonate vials with sealing caps (Nalgene 3138-0050). A ½″ stainless steel ball was also added. The vials were mixed on a vortex mixer before placing in a FinePCR FX4894E rotisserie incubator fitted with FinePCR FX4895FFX sample racks. The temperature was set to 52.5° C., and the incubation time was 5 days.

After incubation, samples for HPLC analysis were prepared as follows. The vials were centrifuged for 10 min. Supernatant samples was diluted five fold with 5 mM sulfuric acid and acidified to a pH below 2 with 40% sulfuric acid. Finally, the samples were filtered through 0.2 μm syringe filters (Whatman GD/X PTFE, 25 mm diameter). Dissolved carbohydrate concentrations were measured by HPLC using an Aminex® HPX-87H column according to the procedure described in NREL/TP-510-42623, January 2008.

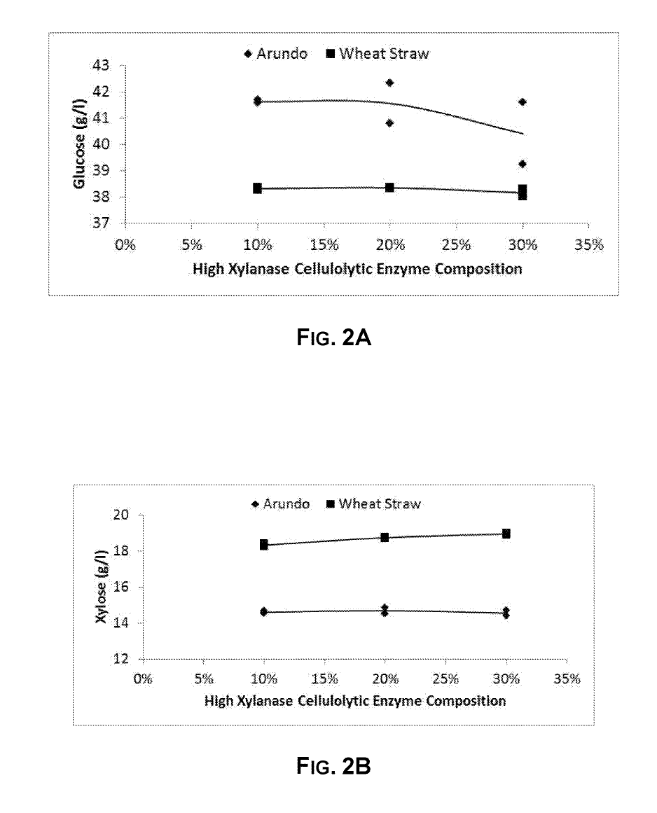

FIG. 2A shows the final glucose concentrations of the supernatant samples. It is seen that an increase of the fraction of XPrep in the total enzyme protein above 10% had little effect on the glucose yield from either substrate. FIG. 2B shows the final xylose concentrations of the supernatant samples. It is seen that for arundo an increase of the fraction of XPrep in the total enzyme protein above 10% had little effect on the xylose yield, but that for wheat straw an increased fraction of XPrep in the total enzyme protein above 10% gives only a very small benefit, approximately 3% increase in xylose yield.

### Example 3

### Improved Hydrolysis of Pre-Treated Wheat Straw in Continuous Reactor by Increased Xylanase Activity

Hydrolysis was performed on pretreated wheat straw prepared as described in Example 1. The following reactions were performed and compared:
Pure batch hydrolysis using CPrepA;
Pure batch hydrolysis using CPrepA:XPrep in a mass dose ratio of 80:20;
First stage CSTR followed by later stage batch reactor hydrolysis using CPrepA; and
First stage CSTR followed by later stage batch reactor hydrolysis using CPrepA:XPrep in a mass dose ratio of 80:20

The pure batch hydrolyses were each performed in a mixed tank reactor filled with a total of 15 kg reaction mass. CPrepA or CPrepA:XPrep (80:20) was added at the start of the reaction. The reaction was performed at 20% dry matter, 50° C. and pH 5.0 for the CPrepA:XPrep (80:20) batch reaction and at 19% dry matter, 50° C. and pH 5.0 for the CPrepA batch reaction. The CPrepA dose in the batch reaction was 4.9% (weight/weight glucan). The CPrepA:XPrep (80:20) dose in the batch reaction was 4.8% (weight/weight glucan).

A first stage CSTR contained a total reaction mass of 150 kg and was operated by discharging 10 kg of hydrolysis reaction material every hour and immediately adding 10 kg of pre-treated wheat straw material and water as 3.2 kg soaked liquid, 4 kg solid stream and 2.8 kg water. At the time of each addition of new material to the CSTR, 45.4 g of CPrepA or 37.5 g of CPrepA plus 7.9 g XPrep was added to the CSTR. The CPrepA:XPrep (80:20) dose in the CSTR+Batch reaction was 5.3% (weight/weight glucan). The CSTR retention time was 15 hour. pH was controlled at 5.2 in the CSTR with additions of 2M sodium hydroxide. The following batch hydrolysis was performed in a Labfors 5 BioEtOH reactor (Infors AG, Switzerland). The reaction in the CSTR was performed at 18% dry matter, 50° C. and pH 5 and the reaction in the Labfors 5 BioEtOH reactor was performed at 18% dry matter, 50° C. and pH 5.0. No additional enzymes were added in the subsequent stage batch hydrolysis.

Following hydrolysis, the total glucose and xylose yields of each of the four reactors were evaluated in terms of glucose yield and xylose yield. Glucose yield is the percent ratio of the amount of glucose in the hydrolyzed mixture to the amount of glucans in the pretreated streams, expressed as glucose equivalents. Glucose equivalents were calculated including insoluble glucans, gluco-oligomers, cellobiose and glucose, present in both the solid and liquid of the lignocellulosic biomass, taking into account the different molecular weights. Equivalently, xylose yield is the percent ratio of the amount of xylose in the hydrolyzed mixture to the amount of xylans in the pretreated streams, expressed as xylose equivalents. Xylose equivalents were calculated including insoluble xylans, xylooligomers, xylobiose and xylose, present in both the solid and liquid of the lignocellulosic biomass, taking into account the different molecular weights.

Table 2 shows glucose and xylose yields at different time points of the enzymatic hydrolysis of pre-treated wheat straw in a continuously stirred tank reactor followed by hydrolysis in batch (CSTR+batch) compared to hydrolysis in only batch (Pure batch) with CPrepA or CPrepA:XPrep (80:20).

TABLE 2

| | CSTR + Batch | | | | CSTR + Batch | | | | Batch | | | Batch | | | |
|---|---|---|---|---|---|---|---|---|---|---|---|---|---|---|---|
| | (CPrepA) | | | | (CPrepA:XPrep (80:20)) | | | | (CPrepA) | | | (CPrepA:XPrep (80:20)) | | | |
| Time (h) | 10 | 25 | 48 | 72 | 15 | 38 | 62 | 75 | 24 | 48 | 60 | 10 | 24 | 48 | 100 |
| glucose | 21 | 29 | 34 | 40 | 21 | 32 | 45 | 47 | 37 | 47 | 50 | 18 | 35 | 46 | 48 |
| yield | | | | | | | | | | | | | | | |
| xylose | 35 | 44 | 49 | 54 | 44 | 45 | 65 | 65 | 50 | 58 | 59 | 42 | 56 | 64 | 64 |
| yield | | | | | | | | | | | | | | | |

The hydrolysis of the CSTR+batch reaction with CPrepA:XPrep (80:20) reached a glucose yield of 47% and a xylose yield of 65% after 75 h of reaction time compared to a glucose yield of 50% and a xylose yield of 59% for the batch hydrolysis with CPrepA after 60 h. The glucose yield was 48% and xylose yield 64% in batch reactor for CPrepA:XPrep (80:20) after 100 h.

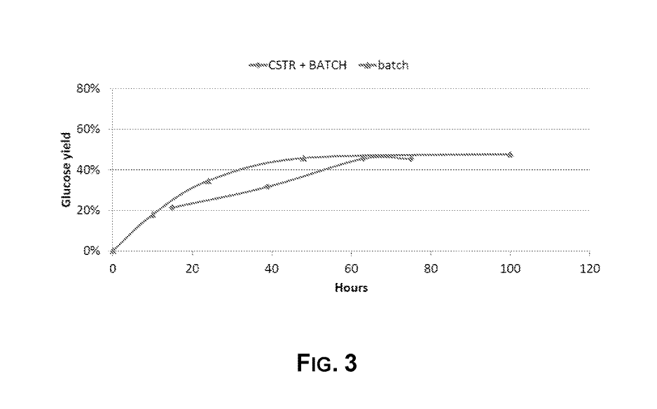

The glucose yield obtained with a first stage hydrolysis in a CSTR, followed by second stage hydrolysis in a batch reactor (CPrepA:XPrep (80:20)) was seen to be similar to a pure batch hydrolysis (CPrepA:XPrep (80:20)). A graph of glucose yield is provided in FIG. 3.

Analytical measurements were performed according to the following standards issued by the National Renewable Energy Laboratory (NREL):

Determination of Structural Carbohydrates and Lignin in Biomass; Laboratory Analytical Procedure (LAP) Issue Date: Apr. 25, 2008; Technical Report NREL/TP-510-42618 Revised April 2008

Determination of Extractives in Biomass; Laboratory Analytical Procedure (LAP) Issue Date: Jul. 17, 2005; Technical Report NREL/TP-510-42619 January 2008

Preparation of Samples for Compositional Analysis; Laboratory Analytical Procedure (LAP) Issue Date: Sep. 28, 2005; Technical Report NREL/TP-510-42620 January 2008

Determination of Total Solids in Biomass and Total Dissolved Solids in Liquid Process Samples; Laboratory Analytical Procedure (LAP) Issue Date: Mar. 31, 2008; Technical Report NREL/TP-510-42621 Revised March 2008

Determination of Ash in Biomass; Laboratory Analytical Procedure (LAP) Issue Date: Jul. 17, 2005; Technical Report NREL/TP-510-42622 January 2008

Determination of Sugars, By products, and Degradation Products in Liquid Fraction Process Samples; Laboratory Analytical Procedure (LAP) Issue Date: Dec. 8, 2006; Technical Report NREL/TP-510-42623 January 2008

Determination of Insoluble Solids in Pretreated Biomass Material; Laboratory Analytical Procedure (LAP) Issue Date: Mar. 21, 2008; NREL/TP-510-42627 March 2008

### Example 4

### Cellulase Assay

An assay of cellulase is based on the enzymatic endo-hydrolysis of the 1,4-β-D-glucosidic bonds in carboxymethylcellulose (CMC). The products of the reaction β-1,4 glucan oligosaccharides are determined colorimetrically by measuring the resulting increase in reducing groups using a 3,5-dinitrosalicylic acid reagent. Enzyme activity is calculated from the relationship between the concentration of reducing groups, as glucose equivalents, and absorbance at 540 nm.

One unit of cellulase activity is defined as the amount of enzyme which produces 1 micro mole glucose equivalents per minute (U) under the conditions of the assay (pH 5.0 and 50° C.). Activity is expressed as U/mg total enzyme.

### Materials

1.0% (w/v solution) Carboxymethylcellulose (CMC) solution in 50 mM sodium citrate buffer, pH 5.0.

3,5-Dinitrosalicylic acid (DNS) solution: 20 g/L DNS; 20 g/L NaOH; 4 g/L phenol; 1 g/L sodium metabisulphite

Glucose standard solution (1 mg/mL).

### Procedure

The enzyme is diluted with 50 mM sodium citrate buffer (pH 5) ranging from 0.2-0.05 ug of enzyme. A glucose standard curve is made using glucose concentrations of 0.06, 0.12, 0.25, 0.5, and 1 mg/mL. In a PCR plate, 50 μL of enzyme solution is mixed with 50 μL of the CMC substrate and incubated at 50° C. in a thermalcycler. The reaction is stopped after 10 min by addition of 80 μL of DNS solution. This is followed by heating at 95° C. for 10 minutes. Then 130 μL of solution is transferred to a flat bottom clear microplate. The optical density is measured at 540 nm for the different samples and standards.

### Example 5

### Xylanase Assay

An assay of xylanase is based on the enzymatic endo-hydrolysis of the 1,4-β-D-xylosidic bonds in birchwood xylan. The products of the reaction β-1,4 xylan oligosaccharides were determined colorimetrically by measuring the resulting increase in reducing groups using a 3,5-dinitrosalicylic acid reagent. Enzyme activity is calculated from the relationship between the concentration of reducing groups, as xylose equivalents, and absorbance at 540 nm.

One unit of xylanase activity is defined as the amount of enzyme which produces 1 micro mole xylose equivalents per minute (U) under the conditions of the assay (pH 5.0 and 50° C.). Activity is expressed as U/mg total enzyme.

### Materials

1.0% (w/v solution) birchwood xylan solution in 50 mM sodium citrate buffer, pH 5.0.

3,5-Dinitrosalicylic acid (DNS) solution: 20 g/L DNS; 20 g/L NaOH; 4 g/L phenol; 1 g/L sodium metabisulphite

Xylose standard solution (1 mg/mL).

### Procedure

The enzyme is diluted with 50 mM sodium citrate buffer (pH 5) ranging from 0.2-0.05 ug of enzyme. A xylose standard curve is made using xylose concentrations of 0.06, 0.12, 0.25, 0.5, and 1 mg/mL. In a PCR plate, 50 μL of enzyme solution is mixed with 50 μL of the birchwood xylan substrate and incubated at 50° C. in a thermalcycler. The reaction is stopped after 10 min by addition of 80 μL of DNS solution. This is followed by heating at 95° C. for 10 minutes. Then 130 μL of solution is transferred to a flat bottom clear microplate. The optical density is measured at 540 nm for the different samples and standards.

### Example 6

### Beta-Xylosidase Assay

An assay of β-xylosidase is based on the enzymatic hydrolysis of the paranitrophenol-β-D-xylopyranoside (pNPX) substrate. The product of the reaction (paranitrophenol) is determined colorimetrically by measuring the resulting increase in absorption at 410 nm under alkaline conditions. Enzyme activity is calculated from the relationship between product and absorbance at 410 nm.

One unit of β-xylosidase activity is defined as the amount of enzyme which produces 1 micro mole pNP product per minute (U) under the conditions of the assay (pH 5.0 and 50° C.). Activity is expressed as U/mg total enzyme.

### Materials

1 mM pNPX solution in 50 mM sodium citrate buffer, pH 5.0.

pNP standard solution (5 mM).

### Procedure

The enzyme is diluted with 50 mM sodium citrate buffer (pH 5) ranging from 4 -0.5 ug enzyme. A pNP standard curve is made using pNP concentrations of 0, 0.03, 0.06, 0.12, 0.25, 0.5, mM. In a microplate, 75 μL of enzyme solution is mixed with 75 μL of the pNPX substrate and incubated at 50° C. The reaction is stopped after 30 min by addition of 100 μL of 0.2 M sodium carbonate solution (pH 11). The optical density is measured at 410 nm for the different samples and standards.

Although the invention has been described with reference to the above examples, it will be understood that modifications and variations are encompassed within the spirit and scope of the invention. Accordingly, the invention is limited only by the following claims.

1. A process of multi-stage saccharification of a lignocellulosic material, the process comprising the steps of:
a) saccharifying a lignocellulosic material in a continuously stirred tank reactor (CSTR) with an enzyme composition comprising a cellulolytic composition and a hemicellulolytic composition in a mass ratio of from about 85:15 to about 65:35 (cellulolytic composition:hemicellulolytic composition) and
b) continuing saccharification in an additional reactor in series with the CSTR without further addition of enzymes to form a hydrolyzate wherein the hydrolyzate has a glucose or xylose yield that is improved as compared to the glucose or xylose yield from a process without addition of such enzyme composition.

2. A process of producing a fermentation product from a lignocellulosic material, the process comprising the steps of:
a) saccharifying the lignocellulosic material, comprising:
1) saccharifying a lignocellulosic material in a continuously stirred tank reactor (CSTR) with an enzyme composition comprising a cellulolytic composition and a hemicellulolytic composition in a mass ratio of from about 85:15 to about 65:35 (cellulolytic composition:hemicellulolytic composition); and
2) continuing saccharification in an additional reactor in series with the CSTR without further addition of enzymes to form a hydrolyzate wherein the hydrolyzate has a glucose or xylose yield that is improved as compared to the glucose or xylose yield from a process without addition of such enzyme composition; and
b) fermenting the hydrolyzate to produce a fermentation product.

3. A process of improving a glucose or xylose yield of saccharification of a lignocellulosic material in a continuously stirred tank reactor (CSTR), the process comprising the steps of:
a) saccharifying a lignocellulosic material in a CSTR with an enzyme composition comprising a cellulolytic composition and a hemicellulolytic composition in a mass ratio of from about 85:15 to about 65:35 (cellulolytic composition:hemicellulolytic composition); and
b) continuing saccharification in an additional reactor in series with the CSTR reactor without further addition of enzymes to form a hydrolyzate wherein the hydrolyzate has a glucose yield or a xylose yield that are improved as compared to the yields from a process without addition of such enzyme composition.

4. The process of claim 1, wherein the pretreated lignocellulosic material has been subjected to a pretreatment method selected from steam explosion and liquid hot water treatment, or a combination thereof.

5. The process of claim 1, wherein the lignocellulosic material has been subjected to a pretreatment selected from chemical pretreatment and mechanical pretreatment, but not subjected to steam explosion or liquid hot water treatment.

6. The process of claim 1, wherein the lignocellulosic material is wheat straw.

7. The process of claim 1, wherein the lignocellulosic material is steam exploded wheat straw.

8. The process of claim 1, wherein the enzyme composition comprises an about 85:15 ratio of cellulolytic composition to hemicellulolytic composition.

9. The process of claim 1, wherein the enzyme composition comprises an about 80:20 ratio of cellulolytic composition to hemicellulolytic composition.

10. The process of claim 1, wherein the enzyme composition comprises an about 70:30 ratio of cellulolytic composition to hemicellulolytic composition.

11. The process of claim 1, wherein the cellulolytic enzyme composition comprises an AA9, a beta-glucosidase, a CBH I and a CBH II.

12. The process of claim 1, wherein the hemicellulolytic enzyme composition comprises a xylanase and a beta-xylosidase.

13. The process of claim 1, wherein the saccharification is performed at a temperature of about 40° C. to about 60° C. or about 50° C. to about 55° C.

14. The process of claim 1, wherein the saccharification is performed at a pH of about 4 to about 6 or about 4.5 to about 5.5.

15. The process of claim 1, wherein the additional reactor is a batch reactor.

16. The process of claim 1, wherein the additional reactor is a CSTR.

17. The process of claim 1, wherein the amount of xylanase in the enzyme composition is about 12.75 U to about 457.5 U per gram of the lignocellulosic material.

18. The process of claim 1, wherein the amount of beta-xylosidase in the enzyme composition is about 0.15 U to about 8.55 U per gram of the lignocellulosic material.

19. The process of claim 1, wherein the amount of cellulase in the enzyme composition is about 3.15 U to about 99.0 U per gram of the lignocellulosic material.
